(19) United States
(12) Patent Application Publication
(10) Pub. No.: US 2008/0057106 A1
(43) Pub. Date: Mar. 6, 2008

(54) Low profile bioactive agent delivery device

(76) Inventors: Signe R. Erickson, Long Beach, CA (US); Laurie R. Lawin, New Brighton, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/897,238

(22) Filed: Aug. 29, 2007

Related U.S. Application Data
(60) Provisional application No. 60/840,890, filed on Aug. 29, 2006.

(51) Int. Cl. A61M 37/00; A61F 2/02; A61F 2/14

(52) U.S. Cl. 424/427; 424/422; 604/175

(57) ABSTRACT

Disclosed are implantable devices that are configured for implantation through tissue or membrane and into an implantation site comprising viscoelastic fluid or non-osseous tissue. In embodiments of the invention, the implantable devices comprise: (a) a nonlinear body member having a direction of extension, a longitudinal axis along the direction of extension, and a proximal portion and a distal portion, wherein at least a portion of the body member deviates from the direction of extension, (b) a retention element at the proximal portion of the body member, the retention element configured to retain the implantable device at the implantation site, the retention element presenting an external profile of no greater than 0.5 mm when the device is implanted in a patient; and (c) a bioactive agent delivery system at the distal portion of the body member, the bioactive agent delivery system comprising one or more bioactive agents. Also disclosed are methods of delivering a bioactive agent to a patient using the devices.

### CROSS-REFERENCE TO RELATED APPLICATIONS

This application claims the benefit of U.S. Provisional Patent Application Ser. No. 60/840,890, filed Aug. 29, 2006, entitled LOW PROFILE BIOACTWVE AGENT DELIVERY DEVICE, the disclosure of which is incorporated herein by reference.

### FIELD OF THE INVENTION

The invention relates to an implantable delivery device for site-specific delivery of one or more bioactive agents to a treatment site within the body.

### BACKGROUND OF THE INVENTION

In recent years, much attention has been given to site-specific delivery of drugs within a patient. Although various drugs have been developed for treatment of a wide variety of ailments and diseases of the body, in many instances, such drugs cannot be effectively administered systemically without risk of detrimental side effects. Site-specific drug delivery focuses on delivering the drugs locally, i.e., to the area of the body requiring treatment. One benefit of the local release of bioactive agents is the avoidance of toxic concentrations of drugs that are at times necessary, when given systemically, to achieve therapeutic concentrations at the site where they are required.

Site-specific drug delivery can be accomplished by injection and/or implantation of a device that releases the drug to the treatment site. Injection of drugs can have limitations, for example, by requiring multiple administrations, increasing risk of complications (such as infection), and patient discomfort. Implantation of a device that delivers drug to the treatment site has therefore gained much interest in recent years.

Further, site-specific drug delivery has been enhanced by technologies that allow controlled release of one or more drugs from an implanted device. Controlled release can relate to the duration of time drug is released from the device, and/or the rate at which the drug is released.

Several challenges confront the use of medical devices that release bioactive agents into a patient's body. For example, treatment may require release of the bioactive agent(s) over an extended period of time (for example, weeks, months, or even years), and it can be difficult to sustain the desired release rate of the bioactive agent(s) over such long periods of time. Further, the device surface is preferably biocompatible and non-inflammatory, as well as durable, to allow for extended residence within the body.

In particular, placement of implantable devices in limited access regions of the body can present additional challenges. Limited access regions of the body can be characterized in terms of physical accessibility as well as therapeutic accessibility. Factors that can contribute to physical accessibility difficulties include the size of the region to be reached (for example, small areas such as eyes, ears and glands), the location of the region within the body (for example, areas that are embedded within the body, such as the middle or inner ear), the tissues surrounding the region (for example, areas such as the eye or areas of the body surrounded by highly vascularized tissue), or the tissue to be treated (for example, when the area to be treated is composed of particularly sensitive tissue, such as areas of the brain).

Factors that can contribute to therapeutic accessibility can be seen, for example, in the delivery of drugs to the eye. Ocular absorption of systemically administered pharmacologic agents is limited by the blood ocular barrier, namely the tight junctions of the retinal pigment epithelium and vascular endothelial cells. High systemic doses of bioactive agents can penetrate this blood ocular barrier in relatively small amounts, but expose the patient to the risk of systemic toxicity. Intravitreal injection of bioactive agents (such as drugs) is an effective means of delivering a drug to the posterior segment of the eye in high concentrations. However, these repeated injections carry the risk of such complications as infection, hemorrhage, and retinal detachment. Patients also often find this procedure somewhat difficult to endure.

Because description of the invention will involve treatment of the eye as an illustrative embodiment, basic anatomy of the eye will now be described in some detail with reference to FIG. 1, which illustrates a cross-sectional view of the eye. Beginning from the exterior of the eye, the structure of the eye includes the iris 38 that surrounds the pupil 40. The iris 38 is a circular muscle that controls the size of the pupil 40 to control the amount of light allowed to enter the eye. A transparent external surface, the cornea 30, covers both the pupil 40 and the iris 38. Continuous with the cornea 30, and forming part of the supporting wall of the eyeball, is the sclera 28 (the white of the eye). The pars plana is a region of the eye approximately 4 mm posterior to the point on the globe where the colored iris 38 meets the white sclera 28. The pars plana encircles the iris and is not constant in width, but rather typically varies between 2-3 mm in diameter around the iris (with the largest diameter of the pars plana typically lying on the temporal size and measuring about 3 mm in width).

The conjunctiva 32 is a clear mucous membrane covering the sclera 28. Within the eye is the lens 20, which is a transparent body located behind the iris 38. The lens 20 is suspended by ligaments attached to the anterior portion of the ciliary body 21. Light rays are focused through the transparent cornea 30 and lens 20 upon the retina 24. The central point for image focus (the visual axis) in the human retina is the fovea (not shown in the figures). The optic nerve 42 is located opposite the lens.

There are three different layers of the eye, the external layer, formed by the sclera 28 and cornea 30; the intermediate layer, which is divided into two parts, namely the anterior (iris 38 and ciliary body 21) and posterior (the choroid 26); and the internal layer, or the sensory part of the eye, formed by the retina 24. The sclera 28 is composed of dense, fibrous tissue and is composed of collagen fiber. Scleral thickness is approximately 1 mm posteriorly near the optic nerve and approximately 0.3 mm anteriorly. At the pars plana, the eye tissues are composed of sclera only; there is no choroidal or retinal tissue layer within this region. For this reason, the a vascular pars plana is typically selected for implantation and/or injection of materials into the interior (vitreous) of the eye.

The lens 20 divides the eye into the anterior segment (in front of the lens) and the posterior segment (behind the lens). More specifically, the eye is composed of three chambers of fluid: the anterior chamber 34 (between the cornea 30 and the iris 38), the posterior chamber 36 (between the iris 38 and the lens 20), and the vitreous chamber 22 (between the lens 20 and the retina 24). The anterior chamber 34 and posterior chamber 36 are filled with aqueous humor whereas the vitreous chamber 22 is filled with a more viscous fluid, the vitreous humor.

The vitreous chamber 22 is the largest chamber of the eye, consisting of approximately 4.5 ml of fluid. The vitreous chamber is filled with a transparent gel composed of a random network of thin collagen fibers in a highly dilute solution of salts, proteins and hyaluronic acid (the vitreous humor comprises approximately 98% water).

### SUMMARY OF THE INVENTION

The present invention provides devices and methods for providing one or more bioactive agents to a treatment site within a patient's body in a manner that presents a low external profile of the device when implanted within a patient. The invention can provide particular advantages when used to deliver bioactive agent(s) to the treatment site in a manner that minimizes damage and/or interference with body tissues and processes, particularly to limited access regions of the patient's body.

The invention relates to an implantable bioactive agent delivery device comprising a device body member that is in a nonlinear (e.g., coil) configuration upon implantation. Upon implantation, the device body has a direction of extension, a longitudinal axis along the direction of extension, and a proximal and distal portion, wherein at least a portion of the body member deviates from the direction of extension. Generally, the proximal portion of the device includes a retention element. The distal portion of the device includes a bioactive agent delivery system. The bioactive agent delivery system comprises one or more bioactive agents and, optionally, a polymeric matrix. The device is provided with a low profile retention element that presents an external profile of no greater than 0.5 mm when in place at an implantation site within a patient.

The retention element is configured to retain the bioactive agent delivery device at an implantation site during treatment. In these aspects, the retention element can provide anchoring features to the device, so that the device remains at the implantation site for the duration of a treatment course. The retention element is also configured to provide a low external profile when in place at the treatment site. In these aspects, the retention element can minimize impact of the device on tissues in the implantation environment (e.g., tissue at the implantation site and adjacent the implantation site) when the device is emplaced within a patient.

The retention element can be configured in a variety of manners. In one embodiment, the retention element comprises a projection at the proximal end of the device body member, the retention element projecting at an angle from the direction of extension of from about 40 to about 100 degrees. The proximal end of the delivery device has a largest dimension substantially perpendicular to the direction of extension that is no greater than 0.5 mm. Optionally, the projection can include one or more suture holes. In some aspects, the retention element can comprise an extension in a direction parallel to the longitudinal axis of the device. Optionally, the extension can include at least one suture hole. The suture hole(s) can be provided at any location of the retention element. In these aspects, the proximal portion of the delivery device has a largest dimension substantially perpendicular to the direction of extension that is no greater than 0.5 mm. In another embodiment, the retention element is configured as a loop suitable for receiving a suturing element. In these aspects, the retention element can present an external profile of no greater than 0.5 mm when in place at an implantation site within a patient. In still further embodiments, the retention element can comprise part of the nonlinear body member itself (for example, suture holes provided within the nonlinear body member itself).

The low profile implantable devices as described herein provide maximum surface area for delivery of bioactive agent within tissues to be treated, and excellent retention due to contact with tissue (e.g., scleral tissue, when implanted within an eye) at the proximal portion of the device. Importantly, minimizing the profile of the device above the scleral surface can reduce the risk of thinning and/or erosion of surrounding tissues, such as the overlying conjunctival membrane. The advantages of the present invention will be primarily discussed relative to the eye, but it will be appreciated that such advantages translate to other treatment sites within the body in a similar manner.

While the nonlinear structure of the device body member can provide some stabilization against the inner wall of the sclera, the additional retention from the low profile retention element provides secure fixing of the device in the sclera, while minimizing the appearance and the potential physical irritation from the proximal portion of the device to the user. Minimizing the appearance of the device can provide certain benefits, in that the patient being treated is less self-conscious of their appearance and potential need to explain the treatment to acquaintances and friends. This enhances patient acceptance of the treatment. Additionally, the low profile of the device reduces the amount of protrusion of the device itself and/or reduces the amount of tissue at the surface of the eye that is involved in the treatment protocol. This in turn can reduce the potential of irritation caused by the device that a patient may perceive, whether real or imagined. The patient therefore may be less likely to rub or otherwise contact the portion of the eye where the device is implanted.

In accordance with principles of the invention, the device is typically implanted through the pars plana of the scleral wall. At the pars plana region, the scleral wall is approximately 0.5 mm in thickness. In some aspects, the implantable device of the invention is configured to include a corresponding distance between the first deviation from the longitudinal axis and the distal face of the lateral projection of the retention element, to provide a secure fit of the device at the implantation site. For example, when the device is provided in a coil configuration, the device can be configured to include a distance between the first coil turn and the distal face of the lateral projection of the retention element that is approximately 0.5 mm (or slightly less) to provide securement of the device at the implantation site. Generally, a large surface area retention element can provide maximum stability of the device at the implantation site, limiting tendency of the distal portion of the device to tilt within the vitreous of the eye. In order to balance this stability with the desire for a small profile retention element, the retention element can be provided with a diameter of about 2 mm.

Thickness of the retention element can be selected to provide anchoring of the device while minimizing the external profile of the device at the implantation site. In some aspects, the thickness of the retention element is about 0.5 mm or less. The shape of the retention element can also be configured to enhance securement of the device while minimizing external profile of the device and/or tissue irritation within the implantation environment. In these aspects, the retention element can be provided as a smooth element, without sharp edges. The retention element can be provided in any shape, and in some aspects can be contoured with the curvature of the globe of the eye.

In some aspects, the retention element can be embedded within scleral tissue. For example, a tunneling, partial thickness dissection of the scleral tissue can be performed in conjunction with implantation of the device in the eye. In these aspects, the device will provide an external profile that is imperceptible or nearly imperceptible from the exterior of the eye.

In further aspects, the retention element can be configured as a suture loop or “eye.” In these aspects, the device can be fully implanted within the vitreous and sutured against the inner scleral wall of the eye. Alternatively, the suture loop can be embedded (partially or wholly) within scleral tissue, when desired.

Various known implantable devices for treatment of the eye have recognized the size constraints presented by implantation of devices within the eye (such as limits based upon volume of the vitreous and/or avoidance of the central visual field). However, additional factors can impact the configuration of the implantable device. For example, ocular tissues surrounding an implantable device are relatively thin and delicate. It is important to minimize and/or avoid irritation of such tissues. One risk when placing an implantable device in an eye is irritation and/or thinning of tissue within the implantation environment, such as the sclera and/or conjunctiva. Various prior art devices have been configured that include a portion that extends through the sclera, to the exterior of the eye. In these devices, a portion of the device is present on the exterior surface of the eye. These portions that extend to the exterior of the eye can present risk of irritation and/or thinning of such tissues within the implantation environment.

In contrast, the invention can provide devices that are capable of being completely implanted within an implantation site. In these aspects, the devices are not part of a larger system that extends from the body, such as a catheter or other drug delivery system that includes portions exterior to the body (such as a drug source). The implantable bioactive agent delivery devices of the invention can provide a therapeutically significant amount of bioactive agent that is suitable for treatment of a desired disease or disorder within a relatively small size. This can be accomplished by virtue of the configuration of the bioactive agent delivery portion of the device, which can maximize bioactive agent delivery surface area while minimizing device displacement volume within the implantation site. This can be particularly beneficial for implantation sites that comprise small anatomical regions, such as the eye, inner ear, sinuses, and the like. Moreover, aspects of the invention can provide a device that is capable of being stably retained in place at an implantation site by virtue of a retention element. Optionally, other portions of the device can further assist in retaining the device in place at the implantation site. Finally, aspects of the invention provide a device that is easily removable from the patient, if desired. Thus, for example, upon completion of a therapeutic treatment, the device can be removed by the surgeon. This can allow for additional implantation of devices, if desired, at other points in time.

In some aspects, the device can provide significant benefits by virtue of residing completely within the patient's body upon implantation. In these aspects, the device does not compromise a surface of the patient's body that presents a barrier against infection. This can reduce risk of infection and other complications that can arise when a device is implanted within a patient. Further, risk of discomfort is lessened.

In further aspects, the invention provides methods for delivering bioactive agent to a patient, the methods comprising steps of:
(a) inserting a bioactive delivery device through tissue or membrane and into an implantation site comprising viscoelastic fluid or non-osseous tissue, the device comprising:
   (i) a nonlinear body member having a direction of extension, a longitudinal axis along the direction of extension, and a proximal portion and a distal portion, wherein at least a portion of the body member deviates from the direction of extension,
   (ii) a retention element at the proximal portion of the body member, the retention element configured to retain the implantable device at the implantation site, the retention element presenting an external profile of no greater than 0.5 mm when the device is implanted in a patient; and
   (iii) a bioactive agent delivery system at the distal portion of the body member, the bioactive agent delivery system comprising one or more bioactive agents,
(b) maintaining the device at the implantation site to provide a therapeutically effective amount of the bioactive agent to the patient.

The method can involve inserting the device into a patient eye. Optionally, the method can further include a step of making an incision in the patient body prior to inserting the bioactive agent delivery device. In some embodiments, the bioactive agent delivery device is inserted by rotating the device into the implantation site until the retention element rests within the tissue or membrane. When implanted into the eye, for example, the method can comprise inserting the device through scleral tissue of a patient eye and rotating the device until the retention element rests within the scleral tissue. Alternatively, the device can be rotated until the retention element passes into a vitreous of the eye. Optionally, the method can further include a step of securing the retention element to the tissue or membrane (for example, by suturing). Such securement can be to scleral tissue of the eye. When the retention element is placed within the vitreous of the eye, the retention element can be secured to scleral tissue at a surface within the vitreous of the eye. Optionally, the device can be removed once a desired treatment course is completed.

These and other aspects and advantages will now be described in more detail.

### BRIEF DESCRIPTION OF THE DRAWINGS

The accompanying drawings, which are incorporated in and constitute a part of this specification, illustrate several aspects of the invention and together with the description of the preferred embodiments, serve to explain the principles of the invention. A brief description of the drawings is as follows:

FIG. 1 is an illustration of a cross-sectional view of the eye.

FIG. 2 is a perspective view of a prior art bioactive agent dispensing device.

FIG. 3 is a perspective view of an embodiment of a bioactive agent delivery device of the invention.

FIG. 4 is a perspective view of another embodiment of a bioactive agent delivery device of the invention.

FIG. 5 is a perspective view of another embodiment of a bioactive agent delivery device of the invention.

FIG. 6 is a perspective view of another embodiment of a bioactive agent delivery device of the invention.

### DETAILED DESCRIPTION OF THE INVENTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

Certain terms will be used herein to describe features of the invention. As used herein, the “external profile” of the implantable device refers to the profile of the implantable device above the external scleral surface (i.e., exterior to the patient eye). The profile above the scleral surface can be described in terms of relative height from the external surface of the sclera, displacement volume, and/or surface area above the external surface of the sclera. The relative height of the device can be measured perpendicularly from the external surface of the sclera and is represented in mm. The displacement volume of the implantable device above the sclera can be represented in cubic mm (mm³). The surface area of the device above the external surface of the sclera can be represented as square mm (mm²) or as a function of device diameter (mm).

In some aspects, the external profile can be described as providing a shape or contour that approximates the surface contour of the implantation site (e.g., the eye). In these aspects, the portion of the device configured as the retention element is provided with a contour or shape that approximates tissues at that portion of the body. In the eye, for example, the retention element can be shaped or contoured to follow the natural curvature of the external surface of the eye. In some aspects, this contour can be envisioned as a “mushroom” shape that provides a domed shape and includes a rounded periphery.

The term “implantation site” refers to the site within a patient's body at which the implantable device is placed according to the invention. The term “incision site” refers to the area of the patient's body (for example, the scleral tissue of the eye, or the skin and transdermal area) at which an incision or surgical cut is made to implant the device according to the invention. The incision site includes the surgical cut, as well as the area in the vicinity of the surgical cut, of the patient. The implantation site is thus located within the patient, interior to an incision site. The “implantation environment” includes the implantation site and tissues surrounding the implantation site, such as the incision site, as well as tissues adjacent to and surrounding the incision site. In turn, a “treatment site” includes the implantation site as well as the area of the body that is to receive treatment directly or indirectly from a device component. For example, bioactive agent can migrate from the implantation site to areas surrounding the device itself, thereby treating a larger area than simply the implantation site.

The displacement volume of the implantable device within the implantation site (e.g., vitreous) can be described in relation to the bioactive agent delivery portion of the device, as well as any portion of the device that does not deliver bioactive agent, so long as the device portion is located within the implantation site.

In turn, the displacement volume of the implantable device within the incision site can be described in relation to the retention element (and any other portion of the device), which resides within at least a portion of the incision site.

In some aspects, the invention further relates to methods for treating a mammalian organism to obtain a desired physiological or pharmacological effect. The method involves administering the bioactive agent delivery device to the mammalian organism and allowing the bioactive agent effective in obtaining the desired physiological or pharmacological effect to depart from the device. The term “administering,” as used herein, means positioning, inserting, implanting, or any other means for exposing the device to a mammalian organism. The route of administration can depend upon a variety of factors, including, for example, the type of response or treatment desired, type of bioactive agent (or agents) to be delivered to the patient, and/or the desired site of administration. One illustrative administration method is to insert the device into the implantation site within a patient and suture it into place. In ocular applications, the method can involve inserting the device through a surgical procedure into the vitreous of the eye, followed by suturing the device in place.

Limited access regions of the body include areas of the body that are difficult to access physically and/or therapeutically. Illustrative limited access regions include the eye, ear, spinal chord, brain, joints, sinuses, glands, and the like.

The implantable devices of the invention can be implanted into a viscoelastic fluid or non-osseous tissue. In exemplary modes of practice, the implantable devices are implanted into a portion of the eye, for example, the vitreous chamber.

The implantable devices can be implanted into a supple body tissue and/or body compartment that includes a gel-like biological material. For example, the device can be delivered to an implantation site comprising non-osseous tissue or a body compartment that includes non-osseous biological material. “Non-osseous” refers to tissue or biological material that is not connective tissue having a matrix that consists of collagen fibers and deposited calcium salts in the form of an apatite.

In some aspects, the implantable devices of the invention can be implanted through tissue or membrane and into a viscoelastic fluid. In these aspects, at least a portion of the device, when implanted, contacts the tissue or membrane (e.g., tissues at the incision site), and at least a portion of the device resides within viscoelastic fluid.

In illustrative embodiments, the implantable devices are configured for implantation within the eye. Typical implantation procedures involve advancing the distal portion of the device by rotational movement into the vitreous of the eye. In many cases, in order for the implantable device to be advanced into the vitreous, it is first advanced through scleral tissue, or scleral and conjunctival tissues of the eye.

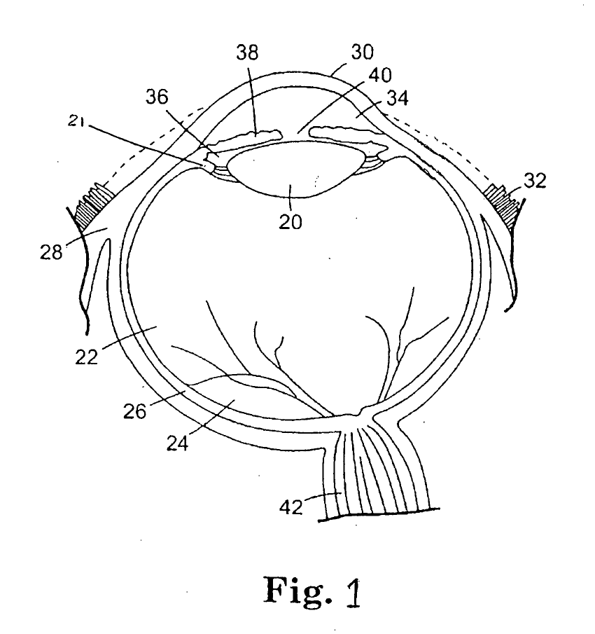

As mentioned, the vitreous chamber is the largest chamber of the eye and contains the vitreous humor or vitreous. In reference to FIG. 1, the vitreous is bound interiorly by the lens 20, posterior lens zonules and ciliary body 21, and posteriorly by the retinal cup.

The vitreous is a transparent, viscoelastic gel that is 98% water and has a viscosity of about 2-4 times that of water. The main constituents of the vitreous are hyaluronic acid (HA) molecules and type II collagen fibers, which entrap the HA molecules. The viscosity is typically dependent on the concentration of HA within the vitreous. The vitreous is traditionally regarded as consisting of two portions: a cortical zone, characterized by more densely arranged collagen fibrils, and a more liquid central vitreous.

Therefore, in some aspects, the invention provides implantable devices configured for implantation into an implantation site within the body, the implantation site comprising a gel-like material, such as viscoelastic gel.

In many aspects of the invention, ocular implantation is performed by rotatably inserting the device into the vitreous. In some aspects, the device can be inserted through the scleral tissue through a penetration in the scieral tissue (trans-scleral insertion) caused by a sharp distal end of the device. Alternatively, in other aspects, the device can be implanted into the vitreous through a sclerotomy previously made in the eye. In these latter aspects, the distal end of the device need not be provided with a sharp configuration, but rather can optionally be provided with a rounded or otherwise bullet-shaped configuration.

In many cases, as indicated, the device is first advanced through a scleral region of the eye. The sclera forms the principal part of the outer fibrous coat of the eye and functions to both protect the intraocular contents and maintain the shape of the globe when distended by intrinsic intraocular pressure (IOP). The sclera proper is relatively a vascular and generally appears white externally. The viscoelastic nature of the sclera (tensile strength, extensibility and flexibility) allows only limited distension and contraction to accommodate minor variations in IOP. The sclera includes connective tissue comprised primarily of collagen (mostly types I and III). The sclera is thickest posteriorly (approximately 1 mm) and thinnest (0.3-0.4 mm) behind the insertions of the aproneurotic tendons of the extraocular muscles. It is covered by the facia bulbi posteriorly and conjunctiva anteriorly. The three histological layers of the sclera, proceeding from the interiormost layer towards the exterior of the globe, are the lamina fusca, stroma and episclera.

Therefore, in some aspects, the invention provides implantable devices configured for implantation through tissue or membrane comprising connective tissue or tissue that includes collagen as a primary component. In some aspects, the tissue or membranes can have a thickness in the range of about 0.2 mm to about 1.0 mm.

In yet further aspects, the invention provides implantable devices comprising a proximal portion and a distal portion, the proximal portion configured to anchor the device at the implantation site (e.g., by anchoring the device in or at a tissue or membrane comprising connective tissue or tissue that includes collagen as a primary component), the distal portion configured to reside within an implantation site within a patient's body. In some embodiments, the implantation site comprises a gel-like material, such as viscoelastic gel. The distal portion includes a bioactive agent delivery system configured to deliver bioactive agent to a treatment site that includes the implantation site within the patient. For example, when the device is configured for implantation within the eye, the device is positioned such that the portion of the implantable device that delivers bioactive agent to the eye chamber is positioned near the posterior segment of the eye.

Thus, in some aspects, the invention provides implantable devices comprising a proximal portion and a distal portion, wherein each portion is configured to reside within a distinct environment within a patient body. In some aspects, the inventive implantable devices comprise a proximal portion configured to reside at or within a tissue or membrane comprising connective tissue or tissue that includes collagen as a primary component, and a distal portion configured to residue within an implantation site (such as an implantation site that includes a gel-like biological material).

In additional aspects, when the implantable device is used to deliver bioactive agent(s) to the eye, the device is preferably designed for insertion through a small incision that requires few or no sutures for scleral closure at the conclusion of the surgical procedure. As such, the device is preferably inserted through an incision that is no more than about 1 mm in cross-section, for example, in the range of about 0.25 mm to about 1 mm in diameter, or about 0.5 mm or less in diameter.

In accordance with some aspects of the invention, the implantable devices can provide a therapeutically effective amount of bioactive agent to a treatment site, while minimizing the impact of implantation and residence of the device within the patient. In some aspects, the implantable devices can be provided with as much as 1 mg of a particular bioactive agent for delivery to a patient. As discussed herein, the implantable devices can be inserted through a small incision and can be configured to present a low external profile once implanted.

The inventive devices include a proximal portion, which refers to the portion of the implantable device that, when in use, is at the user end (that is, closer to the interventionalist than the implantation site). The proximal portion includes a retention element that is directed towards the exterior of a patient. In accordance with the invention, the retention element functions to stabilize the device at the implantation site. In some aspects, the retention element can contribute to an overall low external profile implantable device that minimizes tissue disruption and/or irritation during implantation and use of the device. Optionally, the retention element can facilitate removal of the device, if desired.

The inventive devices further include a distal portion that, when in use, is intended to be located at an implantation site within a patient. The distal portion includes a nonlinear shape, typically a coil or helical shape, and further includes a bioactive agent delivery system. The bioactive agent delivery system is utilized to provide bioactive agent to a patient for a desired treatment course. In preferred aspects, the bioactive agent delivery system provides bioactive agent to a patient in a controlled manner. Optionally, the bioactive agent delivery system includes a polymeric matrix to facilitate and/or enhance such controlled delivery of the bioactive agent.

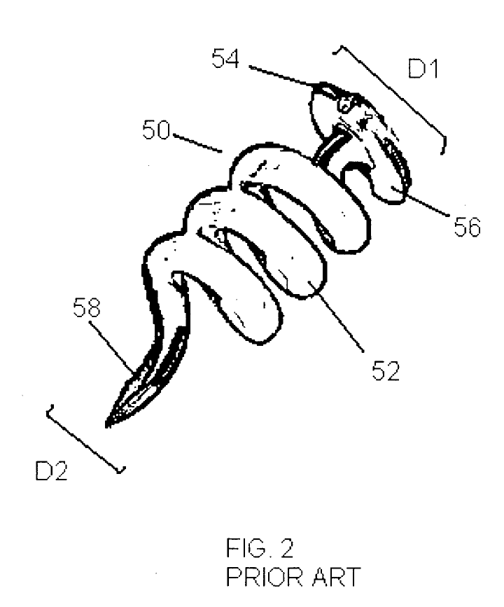

An embodiment of a prior art intraocular bioactive agent delivery system is shown in FIG. 2. Device 50 comprises a nonlinear body 52, having a proximal end 54 with a cap 56 located thereon. Nonlinear body 52 additionally has a distal end 58 that is formed in the shape of a sharp point to assist in insertion of device 50 into an incision in the tissue of a patient at the desired treatment site. As shown in FIG. 2, the cap 56 can have a diameter D¹ that is approximately the same as the outer diameter D² of the coil structure of the helical body 52.

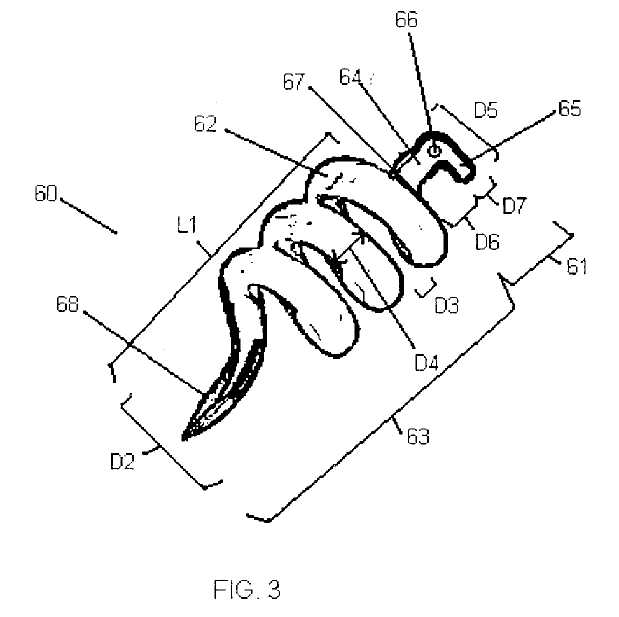

In contrast, the inventive devices include a retention element that does not pass to the exterior of the patient at the incision site. Rather, the inventive devices are configured to provide a low external profile when implanted at an implantation site of a patient. The retention element of the inventive devices can be provided in a variety of configurations. Some illustrative configurations will now be described with reference to FIGS. 3-6.

With reference to the figures, wherein like numerals are used to label like components throughout the several figures, FIG. 3 shows one embodiment of an implantable bioactive agent delivery device of the present invention. The implantable device 60 includes a proximal portion 61 and a distal portion 63. The proximal portion 61 includes retention element 64. Distal portion 63 comprises a body member 62 having a nonlinear shape (represented in a coil configuration in FIG. 3). The distal portion 63 also includes a bioactive agent delivery system (not shown).

As illustrated in FIG. 3, the implantable device 60 is fabricated from a material (such as a wire) having a diameter D⁴. The body member is generally described as having a direction of extension and a longitudinal axis along the direction of extension, wherein at least a portion of the body member deviates from the direction of extension. As shown in FIG. 3, one nonlinear configuration of the body member 62 is a coil configuration. The coil includes a number of deviations from the longitudinal axis (individual turns) that are spaced from one another a distance D³. The outer diameter of the device, D², as measured perpendicularly from the longitudinal axis, can be measured as the largest diameter of an individual deviation (turn) within the coil structure. The body member 62 of the device 60 has a length L¹, as measured in parallel to the longitudinal axis of the device 60. As will become apparent upon review of the present description, each of the dimensions D², D³, D⁴ and L¹ can be individually selected to enhance and/or optimize bioactive agent delivery capabilities of the implantable device 60.

Generally, the proximal portion 61 of the device 60 includes a retention element 64 that has an outer diameter D⁵, as measured perpendicularly to the longitudinal axis. The retention element 64 can be spaced from the body member 62 a distance D⁶ (as discussed below). The retention element itself can have a thickness D⁷, as measured in parallel to the longitudinal axis. As will become apparent upon review of the present description, each of dimensions D⁵, D⁶ and D⁷ can be individually selected to enhance and/or optimize anchoring features of the implantable device 60.

FIG. 3 illustrates a device 60 formed of a material having a circular cross-section. However, it is understood that the material forming the device 60 can be any suitable shape, such as elliptical, square, triangular, and the like. Further, the diameter D⁴ is shown in FIG. 3 as a constant diameter throughout the implantable device 60; however, it is understood that the diameter D⁴ need not be constant throughout the length of the implantable device 60. In some embodiments, for example, the diameter D⁴ can be greater at the proximal portion 61 of the device 60, thereby providing an increased diameter for the retention element of the device. In other embodiments, for example, the diameter D⁴ can be greater within the distal portion 63 of the device 60, for example, to provide increased surface area for the bioactive agent delivery system of the device. Further, the diameter D⁴ can vary within the distal portion 63 of the device 60 as well. Device body member 62 can optionally have any cross-section, and the diameter D⁴ of the material forming the body member 62 may be constant or variable. For example, the diameter D⁴ can progressively increase or decrease from the proximal end to the distal end of the device.

In some aspects of the invention, the diameter D⁴ of the material forming the implantable device 60 is preferably no more than about 0.5 mm, for example, in the range of about 0.25 mm to about 0.5 mm in diameter. When the material forming the implantable device does not have a circular cross-section, the largest dimension of the cross-section can be used to approximate the diameter of the device body member for this purpose, for example, when the device body member cross-section is square.

In some aspects, the nonlinear configuration of body member 62 can be provided by wrapping a material having diameter D⁴ around a hypothetical three dimensional shape (not shown) having a longitudinal axis, wherein the body member proceeds in a direction of extension corresponding to the longitudinal axis of the hypothetical three dimensional shape (and therefore the longitudinal axis of the device). In the embodiment as shown in FIG. 3, the hypothetical three-dimensional shape is a cylinder. The three dimensional shape can be any appropriate shape, such as a cylinder, cone, cube, rectangle, pyramid, and the like. As shown, the major portion of the device has a constant outer diameter D², providing a side view profile similar to a rectangle. The hypothetical three-dimensional shape can have a constant or a variable outer diameter at the respective right sections of the hypothetical three-dimensional shape. If the hypothetical three-dimensional shape is a cylinder, the resulting coil has the appearance of a regular spring with uniform outer diameter and having a side view profile similar to a rectangle. If the hypothetical three-dimensional shape is a cone, the resulting coil is in the shape of a spiral having an increasing (or decreasing) outer diameter from the proximal end to the distal end and having a side view profile similar to a triangle.

The device body member 62 preferably includes at least two, three, four, five, six, seven, eight, nine, ten, or more rotations around the longitudinal axis of the hypothetical three-dimensional shape. Preferably, the device body member has from two to five rotations around the longitudinal axis of the hypothetical three-dimensional shape. The material forming the body member can be any suitable material, for example, a wire, tube, or the like.

Individual deviations within the body member 62 can be provided with a desired spacing D³. In the embodiment as shown in FIG. 3, the turns of the coil are provided in an evenly spaced configuration. Thus, the distance between the individual coils D³ may be substantially uniform from turn to turn. Alternatively the turns may be in progressively increasing or decreasing spaced configuration from the proximal end to the distal end of the body member 62.

The spacing of the individual turns around the central axis of the coiled configuration can be selected to provide an optimum combination of such features as increased surface area for delivery of bioactive agent, overall dimensions of the device, positioning and ease of placement in the body, and the like. For example, when the device body member is provided in the form of a coil that includes two or more rotations around the longitudinal axis, the distance between the individual coils D³ can be selected to be greater than or equal to the scleral thickness. In these aspects, D³ can accommodate the tissue between coil turns during implantation. In an illustrative embodiment, scleral thickness can be approximately 0.5 mm, and D³ can be 0.5 mm or greater. In other aspects, the distance between the individual coils D³ can be selected to be equal to or greater than the diameter D⁴ of the material forming the device body member, which can facilitate coating processes (for example, when a coating is applied to the body member). In one illustrative embodiment of this aspect of the invention, the device body member is formed of a material having a body diameter D⁴ of 0.5 mm, and the distance D³ between each coil of the device body member is at least 0.5 mm.

The outer diameter D² of the body member 62 can be constant along the length of the body member. Alternatively, the diameter D² of the body member 62 can vary along the length of the body member. In an embodiment of the present invention, the outer diameter of the coil rotations decreases toward the distal direction of the device, so that the largest outer diameter is located toward the proximal direction of the body member, and the smallest outer diameter is located toward the distal end of the device body member. In some embodiments, the overall profile of the device body member can be provided in an hourglass shape, alternating perimeter shapes, and the like. The diameter of the coil rotations can be manipulated to provide any desirable configuration. Combinations of different shapes are also contemplated.

As illustrated in FIG. 3, the body member 62 is provided with a length L¹. Typically, the length L¹ will comprise the portion of the device located at the implantation site, i.e., within the patient's body. In some aspects, a proximal region of the length L¹ can be embedded within tissue at the implantation site, for example, within tissue of the incision site. These embodiments will be described in more detail elsewhere. For purposes of illustration, when used to deliver bioactive agent(s) to the eye, the device body member 62 can have a length L¹ that is less than about 1 cm, for example, in the range of about 0.25 cm to about 1 cm. This can, in some embodiments, avoid or reduce risk of the device entering the central visual field.

As illustrated in FIG. 3, the distal portion 63 includes a distal end 68 shown as a sharp distal tip configured to assist in insertion of the implantable device into a patient. The distal end 68 of the body member can include any suitable configuration, depending upon the application of the device and the site of the body at which the device is to be implanted. For example, in some embodiments, the distal end 68 can be rounded. In some embodiments, the distal end 68 of the body member is configured to pierce the body during implantation of the device into the body (e.g., a sharp or pointed tip). In one preferred embodiment, the distal end 68 of the body member has a ramp-like angle. Preferably, the device according to this embodiment can be utilized to make an incision in the body, rather than requiring separate equipment and/or procedures for making the incision site.

The coiled configuration of the device body member 62 can provide an increased surface area for delivery of a bioactive agent to an implantation site as compared to a linear device having the same length and/or outer diameter. This can provide advantages during use of the device, since this configuration allows a greater surface area to be provided in a smaller length and/or outer diameter of the device. For example, in some applications, it can be desirable to limit the length of the device. For example, as will be discussed in more detail herein, it is desirable to limit the length of implants in the eye to prevent the device from entering the central visual field of the eye and to minimize risk of damage to the eye tissues. By providing a device body member having a coiled configuration, the device of the invention has greater surface area (and thus can provide a greater volume of bioactive agent) per length of the device without having to make the cross section of the device, and thus the size of the insertion incision, larger.

In addition, a nonlinear shape of the device (such as a coil) can provide a reduced displacement volume as compared to a device that can have the same outer diameter D² and/or length L¹. To illustrate this aspect of the invention, reference is made to FIG. 3, which illustrates a coil configuration having distance D³ between individual coils, the coils themselves having a diameter D⁴ that is equal to the diameter of the material forming the coil. The coil configuration therefore occupies a volume within the implantation site that is less than a multiple of L¹ and D² (i.e., the device 60 does not occupy the entire space represented by its length times width). This is in contrast to devices that are provided as a solid shape, such as a solid cylindrical device, which would occupy a volume represented by its diameter times its length. Such reduced volume in accordance with the inventive devices can be beneficial, for example, for application in anatomically limited implantation sites within the body, particularly implantation sites that include a viscoelastic fluid.

Additionally, the shape of the device body member in the coiled configuration can assist in reducing unwanted movement of the device and unwanted ejection of the device out of the patient's body, since the shape of the device body member requires manipulation to remove it from an incision. A coil-shaped device body member requires twisting to remove the device from the implantation site.

Optionally, the surface area of the device body member 62 can be enhanced by, for example using a threaded shaft as the device body member or by providing surface configurations such as dimples, pores, raised portions (such as ridges or grooves), indented portions, and the like. The threaded shaft body member configuration (not shown in the figures) is similar to a wood screw configuration. Surface configurations can be formed by micro-etching a device surface, roughening a device surface, and the like techniques. Surface configurations can be beneficial, for example, when the bioactive delivery system comprises a coating on the body member. In these aspects, surface configurations can improve adhesion of a coating to the device body member surface and/or increasing the friction of the device body member relative to the tissue to improve anchoring of the device in the tissue. In an embodiment of the present invention, the device body member has a surface area that is greater than twice the surface area of a like body (i.e. having the same length, diameter and weight) having a smooth surface configuration.

The distal end 68 of the device body member 62 as shown is positioned at the longitudinal axis of implantable device 60. In alternative embodiments, distal end 68 can be spaced from the longitudinal axis, for example at the outer diameter D² of implantable device 60, or at any desired position between the longitudinal axis and outer diameter D².

Turning to the proximal portion 61 of the device 60, FIG. 3 illustrates one embodiment of a retention element 64. As shown, the retention element includes a portion that extends longitudinally along the device length, and a lateral projection 65. The lateral projection 65 of retention element 64 can include a diameter D⁵ measured perpendicularly to the longitudinal axis of the body member. In some aspects, the retention element 64 has a displacement volume of about 0.4 mm³ or less. In one exemplary design, the retention element 64 has a displacement volume of about 0.2 mm³ or about 0.4 mm³. In some aspects, for example, referring to FIG. 3, the lateral projection 65 of the retention element has a diameter D⁵ of about 2 mm or less. In one exemplary design the lateral projection 65 has a diameter D⁵ of about 1 mm to about 2 mm. In some aspects, the retention element has an external profile of no greater than 0.5 mm when in place in the treatment site.

As shown, the low profile retention element 64 includes a lateral projection 65 that projects at an angle from the longitudinal axis of about 90 degrees. In some aspects of the invention, the lateral projection 65 projects at an angle from the longitudinal axis in the range of about 40 to about 100 degrees, or in the range of about 70 to about 100 degrees. The lateral projection 65 illustrated in FIG. 3 is shown as a substantially angular (e.g., rectangular) shaped element. However, it is understood that any suitable shape can be provided for the lateral projection 65, such as a rounded, triangular, and the like shape. In preferred aspects, the retention element 64 and the lateral projection 65 do not include sharp or pointed edges that could injure surrounding tissue upon implantation and/or residence within a patient.

The lateral projection 65 can be provided with a height D⁷ measured along the longitudinal axis of the body member that is selected to provide desirable securement of the implantable device. In some aspects, the height D⁷ is selected to provide suitable securement of the implantable device, while minimizing the external profile of the device. In some embodiments, the height D⁷ of the lateral projection is about 0.5 mm or less, or about 0.4 or less, or about 0.3 mm or less, or about 0.2 mm or less, or in the range of about 1 to about 2 mm.

The lateral projection 65 of the retention element 64 can be spaced from body member 62 a desirable distance. More specifically, the lateral projection 65 can be provided at a distance D⁶ from the proximal face 67 of the first turn of the body member 62. For example, the distance D⁶ is measured between the proximal face 67 of the first turn of the body member 62 and the distal face of the lateral projection 65. In some embodiments, the distance D⁶ corresponds to the thickness of tissue through which the device is implanted. For example, when implanted in the eye, D⁶ can correspond to thickness of scleral tissue at the pars plana region, or about 0.5 mm. In some embodiments, D⁶ is 0.5 mm or less, or 0.4 mm or less, or 0.3 mm or less, or 0.2 mm or less. When the lateral projection 65 projects at an angle other than 90 degrees from the longitudinal axis, the distance D⁶ can be measured from the nearest edge of the lateral projection 65 and the proximal face 67 of the first turn of the body member (i.e., at the smallest angle).

In some aspects, the combined measurement of D⁶ and D⁷ is selected to provide desirable securement of the device, while minimizing the external profile of the implantable device. Thus, the combined measurement of D⁶+D⁷ can be about 1 mm or less, or about 0.9 mm or less, or about 0.8 mm or less, or about 0.7 mm or less, or about 0.6 mm or less, or about 0.5 mm or less.

Optionally, the retention element 64 can be provided with one or more suture holes 66 to assist in securing implantable device 60 to the tissue at the implantation site. In some aspects, two or more suture holes are provided in conjunction with retention element 64. The suture hole(s) are preferably positioned a desirable distance from the edge of the retention element 64, for example, about 0.05 mm to about 0.5 mm inward from any point along the edge of retention element 64. When two suture holes 66 are present, the two suture holes can be provided in a spaced relationship at any position along the retention element 64. It is understood that the positioning of the suture holes 66 is not critical. The suture holes can be provided with any diameter suitable for passage of a suture element therethrough. In some aspects, the suture holes can be provided with a diameter of about 0.1 to about 0.5 mm, as measured perpendicularly to the longitudinal axis of the body member. In some embodiments, the suture holes can extend in a plane perpendicular to the longitudinal axis of the body member, as shown in the figure. The suture hole(s) can be formed in any suitable manner, for example, using a laser, so as to provide cleanly cut opening(s) in the retention element for passage of a suture element.

As illustrated in FIG. 3, the retention element 64 can be positioned at the longitudinal axis of implantable device 60. In alternative embodiments, retention element 64 can be spaced from the longitudinal axis, for example at the outer diameter D² of implantable device 60, or at any desired position between the longitudinal axis and outer diameter D².

The overall dimensions of the implantable device 60 can be selected according to the particular application. For example, the total length of implantable device 60 (including proximal and distal portions) and/or outer diameter D² of the device can be selected to accommodate the particular implantation site. Some factors that can affect the overall dimensions of the implantable device 60 include the potency of any bioactive agent to be delivered (and thus the volume of bioactive agent required, which impacts the surface area of the device, as discussed herein), the location of the implantation site within the body (for example, how far within the body the implantation site is located), the size of the implantation site (for example, a small area such as the eye or inner ear, or a larger area, such as a joint or organ area), the tissue surrounding the implantation site (for example, vascular tissue or hard, calcinous tissue, such as bone), and the like.

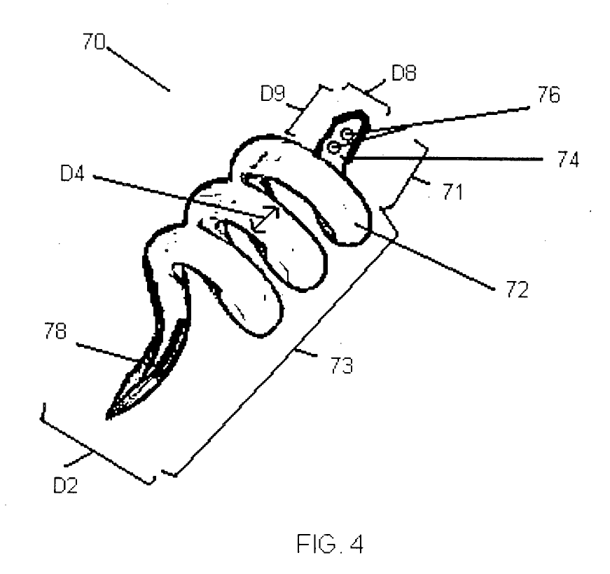

FIG. 4 shows another embodiment of an implantable bioactive agent delivery device in accordance with the present invention. As illustrated, the implantable device 70 includes a proximal portion 71 and a distal portion 73. The proximal portion 71 includes retention element 74. Distal portion 73 comprises a body member 72 having a nonlinear shape (represented in a coil configuration in FIG. 4). The distal portion 73 also includes a bioactive agent delivery system (not shown in the figures).

Distal portion 73 includes a distal end 78 that is shown as a sharp distal tip configured to assist in insertion of the implantable device into a patient. The distal end 78 is similar to the distal end 68 illustrated and described in FIG. 3, and can likewise be provided in a variety of configurations (e.g., sharp, rounded, bullet-shaped, etc.).

Generally, the proximal portion 71 includes a retention element 74 that has an outer diameter D⁸, as measured perpendicularly to the longitudinal axis of the device. The retention element 74 can have a length D⁹, as measured along the longitudinal axis. As will become apparent upon review of the present description, each of dimensions D⁸ and D⁹ can be individually selected to enhance and/or optimize anchoring features of the implantable device 70.

In contrast to the embodiment illustrated in FIG. 3, the implantable device 70 does not include a retention element having a lateral projection. As illustrated in FIG. 4, implantable device 70 includes a retention element 74 that extends in a direction along the longitudinal axis of the device. The retention element 74 thus includes a diameter D⁸, as measured perpendicularly to the longitudinal axis of the device. In some aspects, the retention element 74 has a diameter D⁸ that is equal to or less than the outer diameter D2 of the body member 72 of the device. As described previously, the nonlinear configuration of body member 72 can be provided by wrapping a material having a diameter D⁴ around a hypothetical three-dimensional shape having a longitudinal axis. As illustrated in FIG. 4, the retention element 74 can be formed during this process by providing a portion of the material forming the body member that extends in the longitudinal direction (i.e., a portion of the material forming the body member is not wrapped around the hypothetical three-dimensional shape). In some embodiments, the material forming the retention element 74 can be flattened after formation of the body member 72 and the retention element 74, to thereby provide a diameter D⁸ that is greater than the diameter of the material forming the body member D⁴. In other aspects, the material forming the retention element 74 is maintained in the shape of the original material and thus has a shape similar to the shape of the material forming the body member 72.

In some aspects, the retention element has a diameter D⁸ that is of about 0.5 mm or less. In one exemplary design, the retention element 74 has a diameter D⁸ of about 0.25 mm to about 0.5 mm. In some aspects, the retention element 74 has a displacement volume of about 0.2 mm³ or less. In one exemplary design, the retention element has a displacement volume of about 0.1 mm³ to about 0.2 mm³. In some aspects, the retention element 74 has an external profile of no greater than 0.5 mm² when in place at the treatment site.

As illustrated in FIG. 4, the retention element 74 is provided with a length D⁹, as measured along the longitudinal axis, which is selected to provide desirable anchoring of the device 70 at the implantation site. For example, the length D⁹ can be selected to be approximately equal to the thickness of tissue at the incision site. In ocular applications, for example, the length D⁹ can be selected to be approximately equal to the thickness of the sclera at the pars plana region. In some embodiments, it can be desirable to select the length D⁹ to be less than the thickness of the tissue at the incision site. In some embodiments, the length D⁹ can be on the order of about 0.5 mm or less, or 0.4 mm or less, or 0.3 mm or less, or 0.2 mm or less. The length D⁹ can be selected based upon thickness of the tissue at the incision site (as discussed), and/or the number and/or diameter of any suture holes 76 to be provided in association with the retention element 76.

Optionally, the retention element 74 can be provided with one or more suture holes 76 to assist in securing the implantable device 70 to the tissue at the treatment site. In some aspects, two or more suture holes are provided in conjunction with retention element 74. This configuration affords an exceptionally low external profile. As illustrated in FIG. 4, the diameter D⁸ of the retention element 74 is significantly less than the diameter D² of the body member 72. The relative diameter of the retention element 74 as compared to the body member 72 can be selected based upon such factors as the tissue at the incision site, the implantation site location and/or composition, the overall size of the device, and like factors. For example, the retention element 74 may have a somewhat larger diameter D⁸ than the diameter D² of the body member 72 in order to provide additional material around suture hole 76, or may be somewhat smaller to provide an even lower external profile.

In some aspects, two or more suture holes 76 are provided in conjunction with retention element 74. In these aspects, each suture hole can be positioned a desirable distance from the edge of the retention element. In some embodiments, the suture holes 76 can be provided at 90° angles relative to each other, such that sutures passing through the suture holes 76 of the retention element 74 would be provided at 90° angles relative to each other through the implantable device. It is understood that the positioning of the suture holes 76 is not critical; when multiple suture holes are provided in conjunction with the retention element, the suture holes can be provided in any spaced relationship at any position along the retention element 74. The suture holes can be about 0.1 to about 0.5 mm in diameter, as measured perpendicularly to the longitudinal axis of the body member and preferably extend in a plane perpendicular to the longitudinal axis of the body member, as shown in the figure. The suture hole(s) can be formed in any suitable manner, for example, using a laser, as described elsewhere herein.

The retention element 74 is shown in FIG. 4 as positioned at the longitudinal axis of the implantable device 70. In alternative embodiments, the retention element 74 can be spaced from the longitudinal axis, for example at the outer diameter D² of the device 70, or at any desired position between the longitudinal axis and the outer diameter D².

The discussions above regarding configuration and characteristics of the body member and relative position of various components of the embodiment shown in FIG. 3 apply mutatis mutandis to like parts of the embodiment shown in FIG. 4.

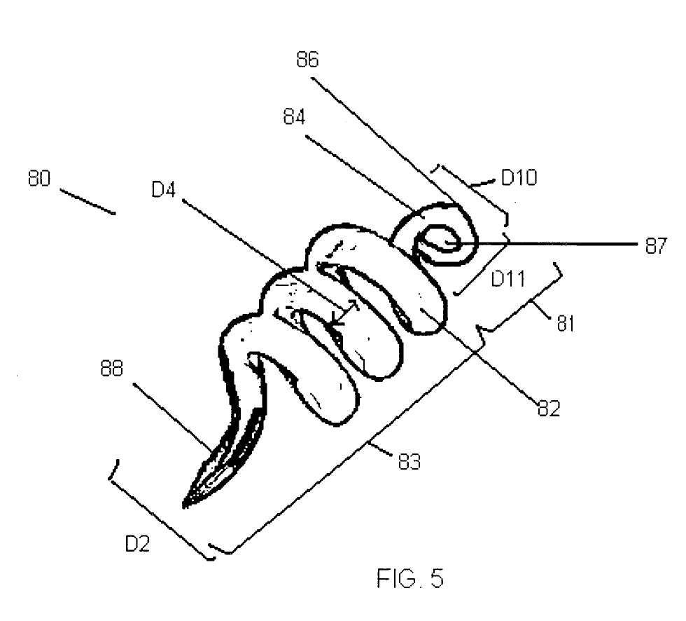

FIG. 5 shows another embodiment of an implantable bioactive agent delivery device of the invention. As illustrated, the implantable device 80 includes a proximal portion 81 and a distal portion 83. The proximal portion 81 includes a retention element 84 in the form of a loop. Distal portion 83 includes a body member 82 having a nonlinear shape, shown in the configuration of a coil. The distal portion 83 further includes a bioactive agent delivery system (not shown).

Distal portion 83 includes a distal end 88 similar to embodiments described previously.

Generally, the proximal portion 81 includes a retention element 84 that has an outer diameter D¹⁰, as measured perpendicularly to the longitudinal axis of the device. The retention element 84 can have a length D¹¹, as measured along the longitudinal axis. Each of dimensions D¹⁰ and D¹¹ can be individually selected to enhance and/or optimize anchoring features of the implantable device 80.

The retention element 84 of implantable device 80 is provided in a loop configuration. The loop defines an aperture 87 through which optional suturing elements can be passed. The use of such optional suturing elements can, in some aspects, enhance anchoring of the device 80 at the implantation site. When utilized, one or more suturing elements can be passed through the aperture 87.

The retention element 84 can be provided with an outer diameter D¹⁰ that is selected to provide desired anchoring of the device 80 at the implantation site. Other factors that can influence the diameter D¹⁰ of the retention element include the tissue at the implantation and/or incision site, the external profile desired once the device is implanted, and the like. In some ocular embodiments, the diameter D¹⁰ is selected to be 2 mm or less, or in the range of about 1 mm to about 2 mm in length.

In accordance with some aspects of the invention, the length D¹¹ of the proximal portion can be selected based upon the tissue at the incision site. In some aspects, the length D¹¹ can be selected such that it is embedded within tissue at the incision site upon implantation of the device 80. Using the eye as an example, the length D¹¹ can be selected so that retention element 84 resides within scleral tissue at the pars plana region upon implantation. In some aspects, D¹¹ can be selected such that the entire retention element 84 resides within scleral tissue. In other aspects, D¹¹ can be selected such that only a portion of retention element 84 resides within scleral tissue upon implantation. When only a portion of the retention element 84 is embedded within tissue upon implantation, the remaining portion of the retention element 84 can reside internally of the patient (e.g., within the implantation site), externally of the patient (thus extended exteriorly of the patient), or both internally and externally of the patient.

In some embodiments, the length D¹¹ is up to 1 mm, as measured along the longitudinal axis of the device 80. For ocular applications, this can correspond to a length that is greater than the thickness of scleral tissue at the implantation site. In some aspects, the length D¹¹ is about 0.5 to about 1 mm in length. In still further aspects, the length D¹¹ is 0.5 mm or less. For ocular applications, this can correspond to a length that is less than the thickness of scleral tissue; in these embodiments, a region of the proximal portion 81 and/or distal portion 83 can be embedded within scleral tissue.

As mentioned, the retention element 84 is configured to form a loop defining an aperture 87. In some embodiments, the device 80 can be secured at an implantation site utilizing one or more suturing elements that pass through the aperture 87. The aperture 87 can be provided with a diameter suitable for passage of one or more suturing elements therethrough. Thus, in some embodiments, a single suturing element can be passed through aperture 87 to secure the device 80 at the implantation site. In other embodiments, multiple suturing elements can be passed through aperture 87, optionally at different angles such that the suturing elements attach to the patient's body at different locations.

In some embodiments, the device 80 is secured at an implantation site without use of suturing elements. In accordance with these aspects of the invention, the retention element 84 can provide anchoring of the device at the implantation site.

The retention element 84 includes a proximal face 86. In some aspects, proximal face 86 is contoured to approximate the surface of tissue at the implantation and/or incision site. For example, when the device is implanted in the eye at the pars plana region of the sclera, the proximal face 86 can be contoured to approximate the external surface contour of the sclera at the pars plana region. In this way, if the device is implanted such that proximal face 86 is at or very near (above or below) the external surface of the sclera, the device will present an external profile that mimics the surface of the body tissue at that point. This can be beneficial in reducing the tendency for tissue irritation and/or thinning at the implantation environment.

The embodiment illustrated in FIG. 5 shows an implantable device 80 having a retention element 84 that presents a small loop outer diameter D¹⁰ that is the same size or smaller than outer diameter D² of body member 82. In an embodiment of the present invention, the small loop outer diameter D¹⁰ less than about half the length of outer diameter D². The perceived profile of implantable device 80 thus is at least as small as small loop outer diameter D¹⁰, and likely smaller depending on the opacity of the tissue surrounding proximal portion 81 of the implantable device 80.

The discussions above regarding configuration and characteristics of the body member and relative position of various components of the embodiment shown in FIG. 3 apply mutatis mutandis to like parts of the embodiment shown in FIG. 5.

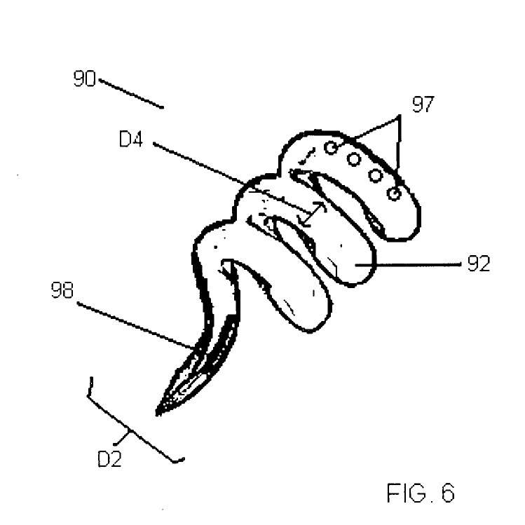

FIG. 6 illustrates a further embodiment of a bioactive agent delivery device in accordance with the inventive concepts. In this embodiment, a portion of the nonlinear body member itself functions as a retention element for the implantable device.

As shown in FIG. 6, an implantable device 90 is provided that includes a body member 92 having a nonlinear shape (represented in a coil configuration in FIG. 6). It will be readily appreciated that the implantable device 90 does not include a separate retention element per se as illustrated in other embodiments described herein. Rather, the function of the retention element is provided by one or more openings 94 provided along a proximal portion of the body member 92. One embodiment, illustrated in FIG. 6, includes one or more openings along the first turn of the body member 92. Openings 94 can be formed as described elsewhere herein.

The number and location of openings 94 can be selected to provide desirable anchoring function at the implantation site. Other factors that can influence the number and/or location of openings 94 include the diameter D² of the body member at the proximal portion, the diameter of the openings 94 themselves, the diameter of the suturing element intended to pass through the openings and thereby secure the device at the implantation site, and the like. In some aspects, one opening is provided. In other aspects, two or more openings are provided, or three or more, or four or more, or five or more.

In these embodiments, the body member 92 of the implantable device 90 itself will be pulled up against the tissue at the incision site. For example, when the device is implanted in the eye, the body member 92 will be pulled up against and contact the scleral tissue of the eye. In some aspects, body member 92 can be partially embedded within scleral upon at the incision site (e.g., scleral tissue).

The discussions above regarding configuration and characteristics of the body member and relative position of various components of the embodiment shown in FIG. 3 apply mutatis mutandis to like parts of the embodiment shown in FIG. 5.

In general, materials used to fabricate the implantable device of the invention are not particularly limited. In some embodiments, the implantable device can be fabricated of a flexible material, so that small movements of the implantable device will not be translated to the implantation site and/or the incision site. In some embodiments, as described in further detail herein, it can be preferable to fabricate at least the distal end of the body member of a rigid, non-pliable material. For example, when the device is designed for implantation in the eye, it is preferable to fabricate the device of a rigid material, to provide improved implant/explant characteristics to the device. In some embodiments it can be preferable to fabricate the implantable device of a material having shape memory and/or superelastic characteristics.

In some embodiments, the implantable device can be fabricated from any suitable material used to manufacture medical devices, such as, for example, stainless steel (for example, 316L); platinum; titanium; and gold; and such alloys as cobalt chromium alloys, nitinol, or the like. In further embodiments, suitable ceramics can be used to fabricate the body member, such as, for example, silicon nitride, silicon carbide, zirconia, alumina, glass, silica, sapphire, and the like. In still further embodiments, the body member can be fabricated of a suitable composite material, such as composite materials commonly used to fabricate implantable devices. Such composite materials can, in some embodiments, provide such advantages as increased strength of the material, as well as increased flexibility. Examples of suitable composite materials include polymers or ceramics (such as high density polyethylene (HDPE), ultra high molecular weight polyethylene (UHMWPE), polymethylmethacrylate bone cement (PMMA), dental polymer matrix (such as crosslinked methacrylate polymers), and glass-ceramics) reinforced with fibers or particulate material (such as carbon fibers, bone particles, silica particles, hydroxyapatite particles, metal fibers or particles, or zirconia, alumina, or silicon carbide particles). Nano-composite materials are also contemplated.

In one embodiment, the body member is fabricated of a non-biodegradable polymer. Such non-biodegradable polymers are well known and can include, for example, oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; and vinyls such as ethylene, propylene, styrene, vinyl chloride, vinyl acetate, and vinylidene difluoride. Examples of condensation polymers include, but are not limited to, nylons such as polycaprolactam, polylauryl lactam, polyhexamethylene adipamide, and polyhexamethylene dodecanediamide, as well as polyurethanes, polycarbonates, polyamides, polysulfones, poly(ethylene terephthalate), polylactic acid, polyglycolic acid, polydimethylsiloxanes, and polyetherketone. Other suitable non-biodegradable polymers include silicone elastomers; silicone rubber; polyolefins such as polypropylene and polyethylene; homopolymers and copolymers of vinyl acetate such as ethylene vinyl acetate 2-pyrrolidone copolymer; polyacrylonitrile butadiene; fluoropolymers such as polytetrafluoroethylene and polyvinyl fluoride; homopolymers and copolymers of styrene acrylonitrile; homopolymers and copolymers of acrylonitrile butadiene styrene; polymethylpentene; polyimides; natural rubber; polyisobutylene; polymethylstyrene; latex; and other similar non-biodegradable polymers.

In some embodiments, the implantable device can be fabricated from a biodegradable material, such as described in U.S. Publication No. 2006/0024350 A1 (Feb. 2, 2006, Varner et al.) or U.S. Publication No. 2006/0034891 A1 (Feb. 16, 2006, Lawin et al.).

The implantable device may optionally be an assembly of two or more components made from different materials. For example, if the distal end of the body member is used to pierce the body during insertion, at least the distal end is preferably fabricated of a rigid, non-pliable material suitable for piercing the body. Such materials are well known and can include, for example, polyimide and similar materials.

The implantable device can be fabricated from a solid material (a material that does not contain a lumen) or a material containing a lumen, as desired. In some embodiments, the lumen can contain a bioactive agent delivery system as described herein.

When included, the lumen(s) can extend along the length of the implantable device or only a portion of the length of the implantable device, as desired. In some embodiments, the lumen(s) can serve as a delivery mechanism for delivery of a desired substance to the implantation site. The substance delivered via the lumen can comprise any of the bioactive agents described herein. The substance delivered via the lumen can be the same or different bioactive agent(s) from that included in a coating composition (when a coating composition is included with the device). Further, the substance can be provided in addition to the bioactive agent of the coating composition, or in place of the bioactive agent. For example, in one embodiment, one or more substances can be delivered via the lumen, and one or more bioactive agents can be provided to the implantation site from the coated composition.

In some embodiments, the lumen can contain a bioactive agent delivery system as described herein. According to these particular embodiments, the body member of the device can be provided with or without a coating on its external surface. In some such embodiments, the lumen can be utilized to deliver the bioactive agent(s) to the implantation site. For example, the lumen can contain the bioactive agent delivery system (bioactive agent(s) and optionally, a polymer material). According to this particular embodiment, the implantable device can be provided with a coating on an external surface comprising the a polymer material only (that is, lacking any bioactive agent). Thus, the bioactive agent is provided to the implantation site in this embodiment principally via the lumen of the body member. In other embodiments, the lumen can include a bioactive agent delivery system (including bioactive agent(s) and optionally, polymeric material), and the implantable device is not provided with a coated composition on its external surface.

The lumen can contain any combination of elements, as desired. For example, in some embodiments, the lumen can include only the bioactive agent to be delivered. In other embodiments, the lumen can include the bioactive agent to be delivered, as well as a polymeric material. The particular combination of elements to be included in the lumen can be selected depending upon the desired application of the device.

When the lumen is to be provided with a bioactive agent and/or polymeric material, the lumen can be filled with the desired bioactive agent and/or polymeric material prior to inserting the device into the body, or after the device has been inserted into the body. When it is desired to fill the device with the substance after insertion into the body, a port can be provided near the proximal end of the implantable device for such purpose. The port is in fluid communication with the lumen(s) of the body member and can also be used for refilling the device with the bioactive agent(s) and/or polymeric material after implantation, when desired.

In further embodiments, when the device includes more than one lumen, the device can include more than one port. For example, each lumen can be in fluid communication with a plurality of ports. These ports are similar to the single port described above. If desired, the lumens and ports can be arranged such that each lumen can be filled with a different material through a corresponding port (for example, each lumen has its own dedicated port). It can be desirable to include more than one lumen when it is desirable to deliver more than one additional material to the implantation site.

In embodiments where it is desired to deliver one or more additional bioactive agents to the implantation site via one or more lumens, the individual lumens can include one or more apertures to allow such delivery. In one embodiment, such apertures are provided at the distal portion of the device. In other embodiments, the apertures are provided along the length of the implantable device, for example, along a length of the body member. The number and size of the apertures can vary depending upon the desired rate of delivery of the bioactive agent (when provided) and can be readily determined by one of skill in the art. The apertures are preferably designed such that the substance to be delivered is slowly diffused rather than expelled as a fluid stream from the device. For example, when the device is implanted in the eye, it is preferable to deliver the substance through slow diffusion rather than expulsion of the substance as a fluid stream, which can damage the delicate tissues of the eye. In some embodiments, the bioactive agent delivery system in contact with the body can provide a particular porosity to the substance and can assist in controlling the rate of diffusion of the substance from the lumen. When included in the device, the particular location of the apertures can be situated so as to deliver the substance at a particular location once the device is implanted into the body.

In some aspects, the material forming the implantable device can be chosen to be permeable (or semi-permeable) to the bioactive agent to be delivered from the lumen. According to this particular embodiment, the material can be chosen depending upon the particular application of the device and the bioactive agent to be delivered and can be readily determined by one of skill in the art. Examples of suitable permeable materials include polycarbonates, polyolefins, polyurethanes, copolymers of acrylonitrile, copolymers of polyvinyl chloride, polyamides, polysulphones, polystyrenes, polyvinyl fluorides, polyvinyl alcohols, polyvinyl esters, polyvinyl butyrate, polyvinyl acetate, polyvinylidene chlorides, polyvinylidene fluorides, polyimides, polyisoprene, polyisobutylene, polybutadiene, polyethylene, polyethers, polytetrafluoroethylene, polychloroethers, polymethylmethacrylate, polybutylmethacrylate, polyvinyl acetate, nylons, cellulose, gelatin, silicone rubbers, porous fibers, and the like. According to these particular embodiments, the material used to fabricate the implantable device can be chosen to provide a particular rate of delivery of the bioactive agent, which can be readily determined by one of skill in the art. Further, the rate of delivery of the bioactive agent can be controlled by varying the percentage of the implantable device formed of the permeable (or semi-permeable) material. Thus, for example, to provide a slower rate of delivery, the implantable device can be fabricated of 50% or less permeable material. Conversely, for a faster rate of delivery, the implantable device can be fabricated of greater than 50% of permeable material. When one or more portions of the implantable device, rather than the whole implantable device, is fabricated of a permeable or semi-permeable material, the location of the permeable or semi-permeable material can be situated so as to deliver the bioactive agent at a particular location once the device is implanted at the implantation site.

As noted above, the devices of the invention include a bioactive agent delivery system for delivery of bioactive agents to a patient in a controlled manner. As discussed herein, one or more bioactive agents can be included in the bioactive agent delivery system. Optionally, the bioactive agent can be provided with a polymeric matrix. The bioactive agent delivery system can be provided in association with a surface of the device (e.g., as a coating on a surface) and/or within a lumen of the device, as desired.

According to the invention, the polymer matrix includes a bioactive agent for sustained delivery of the bioactive agent to a treatment site. As used herein, “bioactive agent” refers to an agent that affects physiology of biological tissue. Bioactive agents usefuil according to the invention include virtually any substance that possesses desirable therapeutic and/or prophylactic characteristics for application to the implantation site.

For ease of discussion, reference will repeatedly be made to a “bioactive agent.” While reference will be made to a “bioactive agent,” it will be understood that the invention can provide any number of bioactive agents to a treatment site. Thus, reference to the singular form of “bioactive agent” is intended to encompass the plural form as well.

Exemplary bioactive agents include, but are not limited to, thrombin inhibitors; antithrombogenic agents; thrombolytic agents (such as plasminogen activator, or TPA: and streptokinase); fibrinolytic agents; vasospasm inhibitors; calcium channel blockers; vasodilators; antihypertensive agents; clotting cascade factors (for example, protein S); anti-coagulant compounds (for example, heparin and nadroparin, or low molecular weight heparin); antimicrobial agents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate, minocycline, doxycycline, vancomycin, kanamycin, cephalosporins such as cephalothin, cephapirin, cefazolin, cephalexin, cephardine, cefadroxil, cefamandole, cefoxitin, cefaclor, cefuroxime, cefonicid, ceforanide, cefitaxime, moxalactam, cetizoxime, ceftriaxone, cefoperazone), geldanamycin and analogues, antifungals (such as amphotericin B and miconazole), and antivirals (such as idoxuridine trifluorothymidine, acyclovir, gancyclovir, interferon, α-methyl-P-adamantane methylamine, hydroxy-ethoxymethyl-guanine, adamantanamine, 5-iodo-deoxyuridine, trifluorothymidine, interferon, adenine arabinoside); inhibitors of surface glycoprotein receptors; antiplatelet agents (for example, ticlopidine); antimitotics; microtubule inhibitors; anti-secretory agents; active inhibitors; remodeling inhibitors; antisense nucleotides (such as morpholino phosphorodiamidate oligomer); anti-metabolites; antiproliferatives (including antiangiogenesis agents, taxol, sirolimus (rapamycin), analogues of rapamycin (“rapalogs”), tacrolimus, ABT-578 from Abbott, everolimus, paclitaxel, taxane, vinorelbine); anticancer chemotherapeutic agents; anti-inflammatories (such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide); non-steroidal anti-inflammatories (such as salicylate, indomethacin, ibuprofen, diclofenac, flurbiprofen, piroxicam); antiallergenics (such as sodium chromoglycate, antazoline, methapyriline, chlorpheniramine, cetrizine, pyrilamine, prophenpyridamine); anti-proliferative agents (such as 1,3-cis retinoic acid); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); mydriatics (such as atropine, cyclopentolate, homatropine, scopolamine, tropicamide, eucatropine, hydroxyamphetamine); sympathomimetics (such as epinephrine); antineoplastics (such as carmustine, cisplatin, fluorouracil); immunological drugs (such as vaccines and immune stimulants); hormonal agents (such as estrogens, estradiol, progesterol, progesterone, insulin, calcitonin, parathyroid hormone, peptide and vasopressin hypothalamus releasing factor); beta adrenergic blockers (such as timolol maleate, levobunolol HCl, betaxolol HCl); immunosuppressive agents, growth hormone antagonists, growth factors (such as epidermal growth factor, fibroblast growth factor, platelet derived growth factor, transforming growth factor beta, somatotropin, fibronectin, insulin-like growth factor (IGF)); carbonic anhydrase inhibitors (such as dichlorophenamide, acetazolamide, methazolamide); inhibitors of angiogenesis (such as angiostatin, anecortave acetate, thrombospondin, anti-VEGF antibody such as anti-VEGF fragment—ranibizumab (Lucentis)); dopamine agonists; radiotherapeutic agents; peptides; proteins; enzymes; nucleic acids and nucleic acid fragments; extracellular matrix components; ACE inhibitors; free radical scavengers; chelators; antioxidants; anti-polymerases; photodynamic therapy agents; gene therapy agents; and other therapeutic agents such as prostaglandins, antiprostaglandins, prostaglandin precursors, and the like.

Another group of useful bioactive agents are antiseptics. Examples of antiseptics include silver sulfadiazine, chlorhexidine, glutaraldehyde, peracetic acid, sodium hypochlorite, phenols, phenolic compounds, iodophor compounds, quaternary ammonium compounds, and chlorine compounds.

Another group of useful bioactive agents are enzyme inhibitors. Examples of enzyme inhibitors include chrophonium chloride, N-methylphysostigmine, neostigmine bromide, physostigmine sulfate, tacrine HCL, tacrine, 1-hydroxymaleate, iodotubercidin, p-bromotetraminsole, 10-(α-diethylaminopropionyl)-phenothiazine hydrochloride, calmidazolium chloride, hemicholinium-3,3,5-dinitrocatechol, diacylglycerol kinase inhibitor 1, diacylglycerol kinase inhibitor II, 3-phenylpropargylamine, N-monomethyl-L-arginine acetate, carbidopa, 3-hydroxybenzylhydrazine HCl, hydralazine HCl, clorgyline HCl, deprenyl HCl, L(−)deprenyl HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-diphenylvalerate hydrochloride, 3-isobutyl-1-methylxanthine, papaverine HCl, indomethacin, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate, R(+) p-aminoglutethimide tartrate, S(−)3-iodotyrosine, alpha-methyltyrosine, L(−)alpha methyltyrosine, D,L(−)cetazolamide, dichlorophenamide, 6-hydroxy-2-benzothiazolesulfonamide, and allopurinol.

Another group of useful bioactive agents are anti-pyretics and antiinflammatory agents. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide. Local anesthetics are substances that have an anesthetic effect in a localized region. Examples of such anesthetics include procaine, lidocaine, tetracaine and dibucaine.

In some embodiments, the bioactive agent delivery system comprises bioactive agent and a polymeric material. Any polymeric material capable of delivering bioactive agents in accordance with the principles of the invention can be utilized. Some illustrative polymeric materials are described herein, without limitation.

In one implementation, a mixture of polymer compositions is used to form the polymeric material. A first polymer component provides an optimal combination of various structural/functional properties, including hydrophobicity, durability, bioactive agent release characteristics, biocompatibility, molecular weight, and availability (and cost). Examples of suitable first polymers include poly(alkyl)(meth)acrylates, and in particular, those with alkyl chain lengths from 2 to 8 carbons, and with molecular weights from 50 kilodaltons to 900 kilodaltons. An example of a particularly preferred first polymer is poly n-butylmethacrylate. Such polymers are available commercially, e.g., from Sigma-Aldrich of St. Louis, Mo., with molecular weights ranging from about 200,000 daltons to about 320,000 daltons, and with varying inherent viscosity, solubility, and form (e.g., as crystals or powder).

A second polymer component for the polymeric material of such embodiments provides an optimal combination of similar properties, and particularly when used in admixture with the first polymer component. Examples of suitable second polymers are available commercially and include poly(ethylene-co-vinyl acetate) having vinyl acetate concentrations of between about 10% and about 50%, in the form of beads, pellets, granules, etc. (commercially available are 12%, 14%, 18%, 25%, 33%). pEVA co-polymers with lower percent vinyl acetate become increasingly insoluble in typical solvents, whereas those with higher percent vinyl acetate become decreasingly durable.

A particularly preferred polymer mixture includes mixtures of poly(butylmethacrylate) (PBMA) and poly(ethylene-co-vinyl acetate) co-polymers (pEVA). This mixture of polymers has proven useful with absolute polymer concentrations (i.e., the total combined concentrations of both polymers in the coating composition), of between about 0.25 and about 70 percent (by weight). It has furthermore proven effective with individual polymer concentrations in the coating solution of between about 0.05 and about 70 weight percent. In one preferred embodiment the polymer mixture includes poly(n-butylmethacrylate) (PBMA) with a molecular weight of from 100 kilodaltons to 900 kilodaltons and a pEVA copolymer with a vinyl acetate content of from 24 to 36 weight percent. In a particularly preferred embodiment the polymer mixture includes poly(n-butylmethacrylate) with a molecular weight of from 200 kilodaltons to 400 kilodaltons and a pEVA copolymer with a vinyl acetate content of from 30 to 34 weight percent. The concentration of the bioactive agent or agents dissolved or suspended in the coating mixture can range from 0.01 to 90 percent, by weight, based on the weight of the final coating composition.

The composition of the present invention used to form the polymeric material can be used to coat an implantable device surface using any suitable means, e.g., by dipping, spraying and the like. The suitability of the coating composition for use on a particular material, and in turn, the suitability of the coated composition can be evaluated by those skilled in the art, given the present description. In turn, the coating thickness of a presently preferred composition will typically be in the range of about 5 micrometers to about 100 micrometers, often from about 7 to 10 micrometers. This level of coating thickness is generally required to provide an adequate density of drug to provide adequate activity under physiological conditions.

Suitable polymeric material in accordance with these embodiments are described, for example, in U.S. Pat. No. 6,214,901 (Chudzik et al.); U.S. Pat. No. 6,344,035 (Chudzik et al.); U.S. Pat. No. 6,890,583 (Chudzik et al.); U.S. Pat. No. 7,008,667 (Chudzik et al.); and U.S. Publication Nos. 2005/0260246 A1 (Chudzik et al.); and 2006/0067968 A1 (Chudzik et al.); and related applications.

Another suitable polymeric material can comprise a crosslinkable macromer system. In accordance with these aspects of the invention, the crosslinkable macromer system can comprise two or more polymer-pendent polymerizable groups and one or more polymer-pendent initiator groups. In a preferred embodiment, the polymerizable groups and the initiator group(s) are pendent on the same polymeric backbone. In an alternative preferred embodiment, the polymerizable groups and initiator group(s) are pendent on different polymeric backbones.

In a first alternative embodiment, the macromer system comprises a polymeric backbone to which are covalently bonded both the polymerizable groups and initiator group(s). Pendent initiator groups can be provided by bonding the groups to the backbone at any suitable time, e.g., either prior to the formation of the macromer (for instance, to monomers used to prepare the macromer), or to the fully formed macromer itself. The macromer system itself will typically comprise only a small percentage of macromers bearing both initiator groups and polymerizable groups. The majority of macromers will provide only pendent polymerizable groups, since the initiator groups are typically sufficient if present at far less than 1:1 stoichiometric ratio with macromer molecules.

In an alternative embodiment, the macromer system comprises both polymerizable macromers, generally without pendent initiator groups, in combination with a polymeric initiator. In either embodiment, the initiator will be referred to herein as a “polymeric initiator”, by virtue of the attachment of such initiator groups to a polymeric backbone. Yet another embodiment includes the macromer system having free (non-polymer bound) initiator molecules.

In an illustrative embodiment, the pendent initiator groups are selected from the group consisting of long-wave ultra violet (LWLTV) light-activatable molecules such as 4-benzoylbenzoic acid, [(9-oxo-2-thioxanthanyl)-oxy]acetic acid, 2-hydroxy thioxanthone, and vinyloxymethylbenzoin methyl ether; visible light activatable molecules; eosin Y, rose bengal, camphorquinone and erythrosin, and thermally activatable molecules; 4,4′ azobis(4-cyanopentanoic) acid and 2,2-azobis[2-(2-imidazolin-2-yl) propane] dihydrochloride. An important characteristic of the initiator group is the ability to be coupled to a preformed macromer containing polymerizable groups, or to be modified to form a monomer that can take part in the macromer synthesis, which is subsequently followed by the addition of polymerizable groups. In such an embodiment, the pendent polymerizable groups are preferably selected from the group consisting of pendent vinyl groups, acrylate groups, methacrylate groups, ethacrylate groups, 2-phenyl acrylate groups, acrylamide groups, methacrylamide groups, itaconate groups, and styrene groups.

In a further embodiment, the polymeric backbone is selected from the group consisting of synthetic macromers, such as polyvinylpyrrolidone (PVP), polyethylene oxide (PEO), and polyethylene glycol (PEG); derivatizable naturally occurring polymers such as cellulose; polysaccharides, such as hyaluronic acid, dextran, and heparin; and proteins, such as collagen, gelatin, and albumin.

The polymeric backbone can be either synthetic or naturally-occurring. Generally, the backbone is one that is soluble, or nearly soluble, in aqueous solutions such as water, or water with added organic solvent (e.g., dimethylsulfoxide) or can be rendered soluble using an appropriate solvent or combination of solvents. Alternatively, the polymeric backbone can be a material that is a liquid under ambient physiological conditions. Backbones for use in preparing biodegradable gels are preferably hydrolyzable under in vivo conditions.

In general, the polymeric backbones can be divided into two categories: biodegradable or bioresorbable, and biostable reagents. These can be further divided into reagents that form hydrophilic, hydrogel matrices and reagents that form non-hydrogel matrices.

Bioresorbable hydrogel-forming backbones are generally naturally-occurring polymers such as polysaccharides, examples of which include, but are not limited to, hyaluronic acid, starch, dextran, heparin, chondroitin sulfate, dermatan sulfate, heparan sulfate, keratan sulfate, dextran sulfate, pentosan polysulfate, and chitosan; and protein (and other polyamino acids), examples of which include but are not limited to gelatin, collagen, fibronectin, laminin, albumin, elastin, and active peptide domains thereof Matrices formed from these materials degrade under physiological conditions, generally via enzyme-mediated hydrolysis.

Bioresorbable matrix-forming backbones are generally synthetic polymers prepared via condensation polymerization of one or more monomers. Matrix-forming polymers of this type include polylactide (PLA), polyglycolide (PGA), polycaprolactone (PCL), as well as copolymers of these materials, polyanhydrides, and polyortho esters.

Biostable hydrogel matrix-forming backbones are generally synthetic or naturally occurring polymers that are soluble in water, matrices of which are hydrogels or water-containing gels. Examples of this type of backbone include polyvinylpyrrolidone (PVP), polyethylene glycol (PEG), polyacrylamide (PAA), polyvinyl alcohol (PVA), and the like.

Biostable matrix-forming backbones are generally synthetic polymers formed from hydrophobic monomers such as methyl methacrylate, butyl methacrylate, dimethyl siloxanes, and the like.

As used herein, the term “polymerizable group” will generally refer to a group that is polymerizable by initiation by free radical generation, most preferably by photoinitiators activated by visible or long wavelength ultraviolet radiation. Preferred polymerizable groups include acrylates, methacrylates, ethacrylates, itaconates, acrylamides, methacrylamide, and styrene. Typically, polymerizable groups are incorporated into a macromer subsequent to the initial macromer formation using standard therrmochemical reactions. Thus, for example, polymerizable groups can be added to collagen via reaction of amine containing lysine residues with acryloyl chloride or glycidyl acrylate. These reactions result in collagen containing pendent polymerizable moieties. Similarly, when synthesizing a macromer for use as described in the present invention, monomers containing reactive groups can be incorporated into the synthetic scheme. For example, hydroxyethylmethacrylate (HEMA) or aminopropylmethacrylamide (APMA) can be copolymerized with N-vinylpyrrolidone or acrylamide yielding a water-soluble polymer with pendent hydroxyl or amine groups. These pendent groups can subsequently be reacted with acryloyl chloride or glycidyl acrylate to form water-soluble polymers with pendent polymerizable groups.

Preferred polymeric initiators are photosensitive molecules that capture light energy and initiate polymerization of the macromers. Other preferred polymeric initiators are thermosensitive molecules that capture thermal energy and initiate polymerization of the macromers. Photoinitiation of the free radical polymerization of macromers of the present invention will generally occur by one of three mechanisms. The first mechanism involves a homolytic alpha cleavage reaction between a carbonyl group and an adjacent carbon atom. This type of reaction is generally referred to as a Norrish type I reaction. Examples of molecules exhibiting Norrish type I reactivity and useful in a polymeric initiating system include derivatives of benzoin ether and acetophenone.

The second mechanism involves a hydrogen abstraction reaction, either intra- or intermolecular. This initiation system can be used without additional energy transfer acceptor molecules and utilizing nonspecific hydrogen abstraction, but is more commonly used with an energy transfer acceptor, typically a tertiary amine, which results in the formation of both aminoalkyl radicals and ketyl radicals. Examples of molecules exhibiting hydrogen abstraction reactivity and useful in a polymeric initiating system, include analogs of benzophenone, thioxanthone, and camphorquinone. When using a polymeric initiator of the hydrogen abstraction variety, pendent tertiary amine groups can be incorporated into the polymeric backbone of the macromer. This will insure that all free radicals formed are polymer-bound.

The third mechanism involves photosensitization reactions utilizing photoreducible or photo-oxidizable dyes. In most instances, photoreducible dyes are used in conjunction with a reductant, typically, a tertiary amine. The reductant intercepts the induced triplet producing the radical anion of the dye and the radical cation of the reductant. Examples of molecules exhibiting photosensitization reactivity and useful in a polymeric initiating system include eosin Y, rose bengal, and erythrosin. Reductants can be incorporated into the polymer backbone, thereby assuring that all free radicals will be polymer-bound.

Thermally reactive polymeric initiators are also useful for the polymerization of macromers to form the shell of the beads. Examples of thermally reactive initiators usable in a polymeric initiating system include 4,4′azobis (4-cyanopentanoic acid) and analogs of benzoyl peroxide. A surprisingly beneficial effect of the use of polymeric initiators to polymerize macromers is the increased efficiency of polymerization exhibited by these polymeric initiators as compared to their low molecular weight counterparts. This increased efficiency is seen in all three photoinitiation mechanisms useful for the polymerization of macromers.

When matrix strength or durability is required for a particular application, high efficiency is again a necessary characteristic of an initiation system. When a matrix-forming system is initiated, the free radical polymerization of the system is propagated until gelation and vitrification of the polymerizing system render the diffusion of the elements of the matrix-forming system too difficult. Therefore, the higher the efficiency of the initiation system, the more complete the polymerization resulting in the formation of stronger, more durable matrices. The polymeric initiation systems described in this invention provide a higher degree of efficiency, without the use of accelerants, than is attainable using nonpolymer-bound, low molecular weight initiators.

In another embodiment, the polymeric initiator comprises a polymeric backbone with pendent initiator groups and pendent reactive or affinity groups. These reactive or affinity groups enable the polymeric initiator to bind to target groups on surfaces of interest. This allows the polymeric initiator to bind to the surface of interest. In this manner, interfacial polymerization of macromers can be accomplished. A solution of polymeric initiator-containing pendent reactive or affinity groups is applied to a surface with target sites. The reactive or affinity groups on the polymeric initiator react with the sites on the surface causing the polymeric initiator to bind to the surface. Excess polymeric initiator can then be washed away. A solution of a polymerizable macromer is then applied to the surface. When light energy in applied to the system, a free radical polymerization reaction is initiated only at the surface of interest. By varying the concentration of the polymerizable macromer and the illumination time, the thickness and crosslink density of the resulting matrix on the surface can be manipulated. Generally, there are two methods by which an initiator group can be incorporated into a polymeric backbone. The first method involves the formation of a monomer that includes the initiator. This can be accomplished readily using standard chemical reactions. For example, the acid chloride analog of an initiator can be reacted with an amine-containing monomer, to form a monomer that contains the initiator. The second method of incorporating initiator groups into a polymeric backbone involves coupling a reactive analog of the initiator with a preformed polymer. For example, an acid chloride analog of an initiator can be reacted with a polymer containing pendent amine groups forming a polymer bearing pendent initiator groups.

Illustrative macromer systems in accordance with these aspects of the invention are described in U.S. Pat. No. 6,007,833 (Chudzik et al.), U.S. Pat. No. 6,156,345 (Chudzik et al.), U.S. Pat. No. 6,410,044 (Chudzik et al.), U.S. Pat. No. 6,924,370 (Chudzik et al.), U.S. Patent App. Publication No. 2005/0136,091, and U.S. patent application Ser. No. 11/475,438 (filed Jun. 27, 2006), and related applications.

In another implementation, one or more non-degradable (durable) polymers is used to form the polymeric material of the bioactive agent delivery system. In an embodiment, the non-degradable polymer includes a plurality of polymers, including a first polymer and a second polymer. When the coating solution contains only one polymer, it can be either a first or second polymer as described herein. As used herein, term “(meth)acrylate” when used in describing polymers shall mean the form including the methyl group (methacrylate) or the form without the methyl group (acrylate).

First polymers of the invention can include a polymer selected from the group consisting of poly(alkyl(meth)acrylates) and poly(aromatic(meth)acrylates), where “(meth)” will be understood by those skilled in the art to include such molecules in either the acrylic and/or methacrylic form (corresponding to the acrylates and/or methacrylates, respectively). An exemplary first polymer is poly(n-butyl methacrylate) (pBMA). Such polymers are available commercially, e.g., from Aldrich, with molecular weights ranging from about 200,000 Daltons to about 320,000 Daltons, and with varying inherent viscosity, solubility, and form (e.g., as crystals or powder). In some embodiments, poly(n-butyl methacrylate) (pBMA) is used with a molecular weight of about 200,000 Daltons to about 300,000 Daltons.

Examples of suitable first polymers also include polymers selected from the group consisting of poly(aryl(meth)acrylates), poly(aralkyl (meth)acrylates), and poly(aryloxyalkyl(meth)acrylates). Such terms are used to describe polymeric structures wherein at least one carbon chain and at least one aromatic ring are combined with acrylic groups, typically esters, to provide a composition. In particular, exemplary polymeric structures include those with aryl groups having from 6 to 16 carbon atoms and with weight average molecular weights from about 50 to about 900 kilodaltons. Suitable poly(aralkyl(meth)acrylates), poly(arylalky(meth)acrylates) or poly(aryloxyalkyl (meth)acrylates) can be made from aromatic esters derived from alcohols also containing aromatic moieties. Examples of poly(aryl(meth)acrylates) include poly(9-anthracenyl methacrylate), poly(chlorophenylacrylate), poly(methacryloxy-2-hydroxybenzophenone), poly(methacryloxybenzotriazole), poly(naphthylacrylate) and -methacrylate), poly(4-nitrophenyl acrylate), poly(pentachloro(bromo, fluoro) acrylate) and -methacrylate), and poly(phenyl acrylate) and -methacrylate). Examples of poly(aralkyl (meth)acrylates) include poly(benzyl acrylate) and -methacrylate), poly(2-phenethyl acrylate) and -methacrylate, and poly(1-pyrenylmethyl methacrylate). Examples of poly(aryloxyalkyl (meth)acrylates) include poly(phenoxyethyl acrylate) and -methacrylate), and poly(polyethylene glycol phenyl ether acrylates) and -methacrylates with varying polyethylene glycol molecular weights.

Examples of suitable second polymers are available commercially and include poly(ethylene-co-vinyl acetate) (PEVA) having vinyl acetate concentrations of between about 10% and about 50% (12%, 14%, 18%, 25%, 33% versions are commercially available), in the form of beads, pellets, granules, etc. pEVA co-polymers with lower percent vinyl acetate become increasingly insoluble in typical solvents, whereas those with higher percent vinyl acetate become decreasingly durable.

An exemplary polymer mixture includes mixtures of pBMA and pEVA. This mixture of polymers can be used with absolute polymer concentrations (i.e., the total combined concentrations of both polymers in the coating material), of between about 0.25 wt. % and about 99 wt. %. This mixture can also be used with individual polymer concentrations in the coating solution of between about 0.05 wt. % and about 99 wt. %. In one embodiment the polymer mixture includes pBMA with a molecular weight of from 100 kilodaltons to 900 kilodaltons and a pEVA copolymer with a vinyl acetate content of from 24 to 36 weight percent. In an embodiment the polymer mixture includes pBMA with a molecular weight of from 200 kilodaltons to 300 kilodaltons and a pEVA copolymer with a vinyl acetate content of from 24 to 36 weight percent. The concentration of the active agent or agents dissolved or suspended in the coating mixture can range from 0.01 to 99 percent, by weight, based on the weight of the final coating material.

Second polymers can also comprise one or more polymers selected from the group consisting of (i) poly(alkylene-co-alkyl(meth)acrylates, (ii) ethylene copolymers with other alkylenes, (iii) polybutenes, (iv) diolefin derived non-aromatic polymers and copolymers, (v) aromatic group-containing copolymers, and (vi) epichlorohydrin-containing polymers.

Poly(alkylene-co-alkyl(meth)acrylates) include those copolymers in which the alkyl groups are either linear or branched, and substituted or unsubstituted with non-interfering groups or atoms. Such alkyl groups can comprise from 1 to 8 carbon atoms, inclusive. Such alkyl groups can comprise from 1 to 4 carbon atoms, inclusive. In an embodiment, the alkyl group is methyl. In some embodiments, copolymers that include such alkyl groups can comprise from about 15% to about 80% (wt) of alkyl acrylate. When the alkyl group is methyl, the polymer contains from about 20% to about 40% methyl acrylate in some embodiments, and from about 25% to about 30% methyl acrylate in a particular embodiment. When the alkyl group is ethyl, the polymer contains from about 15% to about 40% ethyl acrylate in an embodiment, and when the alkyl group is butyl, the polymer contains from about 20% to about 40% butyl acrylate in an embodiment.

Alternatively, second polymers can comprise ethylene copolymers with other alkylenes, which in turn, can include straight and branched alkylenes, as well as substituted or unsubstituted alkylenes. Examples include copolymers prepared from alkylenes that comprise from 3 to 8 branched or linear carbon atoms, inclusive. In an embodiment, copolymers prepared from alkylene groups that comprise from 3 to 4 branched or linear carbon atoms, inclusive. In a particular embodiment, copolymers prepared from alkylene groups containing 3 carbon atoms (e.g., propene). By way of example, the other alkylene is a straight chain alkylene (e.g., 1-alkylene). Exemplary copolymers of this type can comprise from about 20% to about 90% (based on moles) of ethylene. In an embodiment, copolymers of this type comprise from about 35% to about 80% (mole) of ethylene. Such copolymers will have a molecular weight of between about 30 kilodaltons to about 500 kilodaltons. Exemplary copolymers are selected from the group consisting of poly(ethylene-co-propylene), poly(ethylene-co-1-butene), polyethylene-co-1-butene-co-1-hexene) and/or poly(ethylene-co-1-octene).

“Polybutenes” include polymers derived by homopolymerizing or randomly interpolymerizing isobutylene, 1-butene and/or 2-butene. The polybutene can be a homopolymer of any of the isomers or it can be a copolymer or a terpolymer of any of the monomers in any ratio. In an embodiment, the polybutene contains at least about 90% (wt) of isobutylene or 1-butene. In a particular embodiment, the polybutene contains at least about 90% (wt) of isobutylene. The polybutene may contain non-interfering amounts of other ingredients or additives, for instance it can contain up to 1000 ppm of an antioxidant (e.g., 2,6-di-tert-butyl-methylphenol). By way of example, the polybutene can have a molecular weight between about 150 kilodaltons and about 1,000 kilodaltons. In an embodiment, the polybutene can have between about 200 kilodaltons and about 600 kilodaltons. In a particular embodiment, the polybutene can have between about 350 kilodaltons and about 500 kilodaltons. Polybutenes having a molecular weight greater than about 600 kilodaltons, including greater than 1,000 kilodaltons are available but are expected to be more difficult to work with.

Additional alternative second polymers include diolefin-derived, non-aromatic polymers and copolymers, including those in which the diolefin monomer used to prepare the polymer or copolymer is selected from butadiene (CH₂═CH—CH═CH₂) and/or isoprene (CH₂═CH—C(CH₃)═CH₂). In an embodiment, the polymer is a homopolymer derived from diolefin monomers or is a copolymer of diolefin monomer with non-aromatic mono-olefin monomer, and optionally, the homopolymer or copolymer can be partially hydrogenated. Such polymers can be selected from the group consisting of polybutadienes prepared by the polymerization of cis-, trans- and/or 1,2-monomer units, or from a mixture of all three monomers, and polyisoprenes prepared by the polymerization of cis-1,4- and/or trans-1,4-monomer units. Alternatively, the polymer is a copolymer, including graft copolymers, and random copolymers based on a non-aromatic mono-olefin monomer such as acrylonitrile, and an alkyl (meth)acrylate and/or isobutylene. In an embodiment, when the mono-olefin monomer is acrylonitrile, the interpolymerized acrylonitrile is present at up to about 50% by weight; and when the mono-olefin monomer is isobutylene, the diolefin is isoprene (e.g., to form what is commercially known as a “butyl rubber”). Exemplary polymers and copolymers have a molecular weight between about 150 kilodaltons and about 1,000 kilodaltons. In an embodiment, polymers and copolymers have a molecular weight between about 200 kilodaltons and about 600 kilodaltons.

Additional alternative second polymers include aromatic group-containing copolymers, including random copolymers, block copolymers and graft copolymers. In an embodiment, the aromatic group is incorporated into the copolymer via the polymerization of styrene. In a particular embodiment, the random copolymer is a copolymer derived from copolymerization of styrene monomer and one or more monomers selected from butadiene, isoprene, acrylonitrile, a C₁-C₄ alkyl (meth)acrylate (e.g., methyl methacrylate) and/or butene. Useful block copolymers include copolymer containing (a) blocks of polystyrene, (b) blocks of an polyolefin selected from polybutadiene, polyisoprene and/or polybutene (e.g., isobutylene), and (c) optionally a third monomer (e.g., ethylene) copolymerized in the polyolefin block. The aromatic group-containing copolymers contain about 10% to about 50% (wt.) of polymerized aromatic monomer and the molecular weight of the copolymer is from about 300 kilodaltons to about 500 kilodaltons. In an embodiment, the molecular weight of the copolymer is from about 100 kilodaltons to about 300 kilodaltons.

Additional alternative second polymers include epichlorohydrin homopolymers and poly(epichlorohydrin-co-alkylene oxide) copolymers. In an embodiment, in the case of the copolymer, the copolymerized alkylene oxide is ethylene oxide. By way of example, epichlorohydrin content of the epichlorohydrin-containing polymer is from about 30% to 100% (wt). In an embodiment, epichlorohydrin content is from about 50% to 100% (wt). In an embodiment, the epichlorohydrin-containing polymers have a molecular weight from about 100 kilodaltons to about 300 kilodaltons.

Suitable non-degradable polymers and mixtures thereof in accordance with these aspects of the invention are described in U.S. Pat. App. Publication Nos. 2005/0220839, 2005/0220840, 2005/0220841, 2005/0220842, 2005/0220843, 2005/0244459, 2006/0083772 (DeWitt et al.), and related applications.

In another implementation, non-degradable polymers can be used to form the polymeric material of the bioactive agent delivery system, such as those described in U.S. Pat. App. No. 60/703,555, filed Jul. 29, 2005 and entitled “DEVICES, ARTICLES, COATINGS, AND METHODS FOR CONTROLLED ACTIVE AGENT RELEASE OR HEMOCOMPATIBILITY,” and U.S. Patent App. No. 60/733,423, filed Nov. 3, 2005, the contents of which are herein incorporated by reference. As a specific example, non-degradable polymers can include random copolymers of butyl methacrylate-co-acrylamido-methyl-propane sulfonate (BMA-AMPS). In some embodiments, the random copolymer can include AMPS in an amount equal to about 0.5 mol. % to about 40 mol.

Specific embodiments of the copolymer include random copolymers of butyl methacrylate-co-acrylamido-methyl-propane sulfonate (pbma-co-AMPS). In certain embodiments, the random copolymer can include AMPS at about 0.5 to about 30 mol-%, about 1 to about 20 mol-%, or about 2 to about 10 mol-%. In certain embodiments, the random copolymer can include AMPS at about 0.5 to about 40 mol-%, about 20 to about 40 mol-%, about 25 to about 35 mol-%, about 25 to about 30 mol-%, or about 30 mol-%.

An embodiment of a polymer including an effective amount of monomeric unit or monomeric units including polar moieties and at least one second monomeric unit (without charged moiety) can be represented, for example, by Formula A:

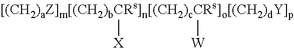

In Formula A: Each [ ] moiety represents a monomeric unit present in the polymer, which can be present in any order, e.g., randomly. Each R^{s} is independently H or CH₃. Each of a, b, c, and d is independently 1-4, 1-3, 1-2, 1, 2, or 3.

Each X and Z is independently a polar moiety. For example, in an embodiment, X can be or include a methyl propane sulfonate moiety (e.g., amido isobutyl sulfonate (—C(O)NHC(CH₃)₂CH₂SO₃H, the pendant moiety in the monomer AMPS)). For example, in an embodiment, X can be or include a methyl propane sulfonate moiety and Z is absent (m=0). In certain embodiments, X can be or include a carboxyl containing moiety, a quaternary ammonium containing moiety, a pyridinium containing moiety, combinations thereof, or the like.

Each W and Y is independently a group that is not a charged moiety. W or Y can be or include, for example, a polar or non-polar moiety. W or Y can be or include, for example, alkyl, aryl, methylene, amide, methyl, alcohol, ether, amide, ester, carbamate, carbonate, combinations thereof, or the like. In an embodiment, W can be or include a —C(O)O—(CH₂)₃CH₃ moiety (the pendant moiety of the monomer butyl methacrylate).

In Formula A, each of m, n, o, and p represents the mole fraction of the corresponding monomeric unit in the polymer, and m+n represents an effective mole fraction. For example, m+n can be about 0.5 to about 35 mol-%, about 1 to about 20 mol-%, or about 2 to about 10 mol-%. By way of further example, m+n can be about 1.5 mol-%, about 3 mol-%, or about 9 mol-%. Either m or n can be zero, but m+n>0. The present polymer can include any of these ranges or amounts not modified by about or any of these quantities individually.

Either o or p can be zero, but o+p>0.

Suitable random copolymers can include polar monomeric units such as water soluble monomeric units. Water soluble monomeric units include those listed as water soluble in the Polymer Handbook (Branderup and Immergut, eds.), 3d Edition (1989) or later, John Wiley and Sons, NY. Suitable random copolymers are soluble in organic solvent.

Suitable random copolymers can include water soluble polar monomeric units such as a water soluble N-substituted acrylamide including a polar or charged substituent (e.g., a cationic or anionic substituent), a water soluble acrylic acid ester including a polar or charged substituent (e.g., a cationic or anionic substituent), a water soluble carboxyl containing monomeric unit, a water soluble quaternary ammonium containing monomeric unit, combinations thereof, or the like. Suitable random copolymers can include an N-substituted acrylamide including a polar substituent such as acrylamido-methyl propane sulfonate (AMPS). Suitable random copolymers can include an N-substituted acrylamide including a charged substituent such as an alkali metal (e.g., sodium) salt of acrylamido-methyl propane sulfonate (AMPS). Suitable random copolymers can include an acrylic acid ester including a polar substituent such as 3-sulfopropyl (meth)acrylate. Suitable random copolymers can include an acrylic acid ester including a charged substituent such as an alkali metal (e.g., sodium) salt of 3-sulfopropyl (meth)acrylate. Suitable random copolymers can include a water soluble N-substituted acrylamide including a cationic substituent such as a water soluble quaternary ammonium substituted acrylamide or methacrylamide.

Suitable random copolymers can include as the second monomeric unit an acrylate or methacrylate. Suitable second monomeric units include N,N-dimethylacrylamide, N,N-diethylacrylamide, N-isopropylacrylamide, N-t-butylacrylamide, N-octylacrylamide, N-cyclohexylacrylamide, N-phenylacrylamide, N-benzylacrylamide, N-methylmethacrylamide, N-ethylmethacrylamide, N,N-dimethylmethacrylamide, N,N-diethylmethacrylamide, alkyl or aryl acrylate, alkyl or aryl methacrylate, vinylmethylether, combinations thereof, or the like. Suitable second monomeric units include a methacrylate, for example, butyl methacrylate.

Suitable polymer backbones including uncharged polar moieties include polyethers (e.g., polyethylene glycol, polypropylene glycol), substituted polyalkyleneimines (e.g., substituted polyethyleneimine), and the like.

Suitable random copolymers include butyl methacrylate-co-acrylamido-methyl-propane sulfonate (pbma-co-AMPS).

The copolymer can include polar monomeric unit at about 0.5 to about 30 mol-%, about 1 to about 20 mol-%, or about 2 to about 10 mol-%. The copolymer can include polar monomeric unit at about 1.5 mol-%, about 3 mol-%, or about 9 mol-%. The copolymer can include second monomeric unit at about 70 to about 99.5 mol-%, about 80 to about 99 mol-%, or about 90 to about 98 mol-%, or about 85 to about 95 mol-%. The copolymer can include second monomeric unit at about 98.5 mol-%, about 97 mol-%, or about 91 mol-%. The present polymer can include any of these ranges or amounts not modified by about or any of these quantities individually.

In an embodiment, the copolymer can include polar monomeric unit at an amount such that the copolymer, when wetted, does not form a hydrogel. In an embodiment, the copolymer can include polar monomeric unit at an amount such that the copolymer, when wetted, does not expand. In an embodiment, the copolymer can include polar monomeric unit at an amount such that the copolymer,.when wetted, does not expand significantly.

The present copolymer composition can be applied to a substrate or device using known methods. For example, the copolymer can be mixed with active agent and solvent and applied to a substrate or device by spraying (e.g., aerosol or ultrasonic), dipping, or with an ultrasonic coater. In some embodiments, the present composition can include a preformed polymer. For example, an active agent may be mixed with a preformed polymer and then deposited on a substrate. The method can include drying the device after applying the copolymer composition.

The copolymer composition can be applied at relative humidity of, for example, about 5% to about 75%, about 5% to about 50%, about 5% to about 35%, or about 5% to about 10%. Although not limiting to the present invention, it is believed that applying the present copolymer composition at a higher relative humidity will increase the rate of active agent release compared to a lower humidity.

Other degradable polymer systems that can be utilized as part of the bioactive agent delivery system include those described in U.S. Patent App. Publication No. 2006/0147491 (DeWitt et al.) and U.S. patent application Ser. No. 11/317,2121 (filed Dec. 22, 2005, DeWitt et al.). These references describe blended and layered polymeric coatings that include a first biodegradable polymer and biodegradable second polymer. The first polymer can be a polyether ester copolymer, such as PEGT/PBT. Other polymers containing ester linkages that are suitable first biodegradable polymers include terephthalate esters with phosphorus-containing linkages, and segmented copolymers with differing ester linkages. In further aspects, the first biodegradable polymer can comprise a polycarbonate-containing random copolymer. In still further aspects, the bioactive agent delivery system of the invention can include the first biodegradable polymer alone (i.e., a second biodegradable polymer is not provided in the bioactive agent delivery system, either as a multiple layer construct, or as a blended polymer material).

Other exemplary multi-block copolymers have a structure according to any of the formulae (1)-(3) as described in EP 1555278:

[—R₁-Q1-R₄-Q2-]ₓ-[R₂-Q3-R₄-Q4-]_{y}-[R₃-Q5-R₄-Q6-]_{z}- (1)

[—R₁—R₂—R₁-Q1-R₄-Q2-]ₓ-[R₃-Q2-R₄-Q1]_{z}- (2)

[—R₂—R₁—R₂-Q1-R₄-Q2-]ₓ-[R₃-Q2-R₄-Q1]_{z}B— (3)

R₁ and R₂ may be amorphous polyester, amorphous polyetherester or amorphous polycarbonate; or an amorphous pre-polymer that is obtained from combined ester, ether and/or carbonate groups. R₁ and R₂ may contain polyether groups, which may result from the use of these compounds as a polymerization initiator, the polyether being amorphous or crystalline at room temperature. However, the polyether thus introduced will become amorphous at physiological conditions. R₁ and R₂ are derived from amorphous pre-polymers or blocks A and B, respectively, and R₁ and R₂ are not the same. R₁ and R ₂ may contain a polyether group at the same time, but it is preferred that only one of them contains a polyether group. “z” is zero or a positive integer. R₃ is a polyether, such as poly(ethylene glycol), and may be present (z≠0) or not (z=0). R₃ will become amorphous under physiological conditions. R₄ is an aliphatic C₂-C₈ alkylene group, optionally substituted by a C₁-C₁₀ alkylene, the aliphatic group being linear or cyclic, wherein R₄ is preferably a butylene, —(CH₂)₄-group, and the C₁-C₁₀ alkylene side group may contain protected S, N, P or O moieties. “x” and “y” are both positive integers, which are both preferably at least 1, whereas the sum of “x” and “y” (x+y) is preferably at most 2000, more preferably at most 500, most preferably at most 200. Q1-Q6 are linking units obtained by the reaction of the pre-polymers with the multifunctional chain-extender. Q1-Q6 are independently amine, urethane, amide, carbonate, ester or anhydride. The event wherein all linking groups Q are different being rare and is not preferred.

Other illustrative degradable polymeric material that can be utilized for the bioactive agent delivery system is described in U.S. Patent Application Publication No. US 2005/0255142 A1 (Chudzik et al.), and U.S. patent application Ser. Nos. 11/271,213, 11/271,237, 11/271,238, and 60/719,466 and 60/791,086, and related applications. Generally, these references describe biodegradable polysaccharides that can be associated with each other to form a matrix. The biodegradable polysaccharides are non-synthetic (natural) and include polysaccharide and/or polysaccharide derivatives that are obtained from natural sources, such as plants or animals. Exemplary natural biodegradable polysaccharides include maltodextrin, amylose, cyclodextrin, polyalditol, hyaluronic acid, dextran, heparin, chondroitin sulfate, dermatan sulfate, heparan sulfate, keratan sulfate, dextran, dextran sulfate, pentosan polysulfate, and chitosan.

In preparing polymeric material in accordance with these aspects, a plurality of natural biodegradable polysaccharides are crosslinked to each other via coupling groups that are pendent from the natural biodegradable polysaccharide (i.e., one or more coupling groups are chemically bonded to the polysaccharide). In some aspects, the coupling group on the natural biodegradable polysaccharide is a polymerizable group. In a free radical polymerization reaction the polymerizable group can crosslink natural biodegradable polysaccharides together in the composition, thereby forming a natural biodegradable polysaccharide matrix.

In yet another implementation, a blend of two or more poly(ester amide) polymers (PEA) polymer can be used to form the polymeric material. Such polymers can be prepared by polymerization of a diol (D), a dicarboxylic acid (C), and an alpha-amino acid (A) through ester and amide links in the form (DACA)ₙ. An example of a (DACA)ₙ polymer is shown in the formula below:

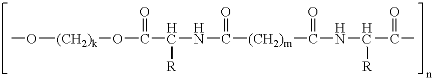

wherein k=2-12,
m=2-12, and
R═CH(CH₃)₂, CH₂CH(CH₃)₂, CH(CH₃)CH₂CH₃, (CH₂)₃CH₃, CH₂C₆H₅, or (CH₂)₂SCH₃,

wherein the blend comprises a PEA polymer wherein R═CH₂CH(CH₃)₂ or a PEA polymer wherein R═CH₂C₆H₅.

Suitable amino acids include any natural or synthetic alpha-amino acid, preferably neutral amino acids. Diols include any aliphatic diol, including alkylene diols such as HO—(CH₂)ₖ—OH (i.e., non-branched), branched diols (e.g., propylene glycol), cyclic diols (e.g., dianhydrohexitols and cyclohexanediol), or oligomeric diols based on ethylene glycol (e.g., diethylene glycol, triethylene glycol, tetraethylene glycol, or poly(ethylene glycol)s).

Suitable dicarboxylic acids may be any aliphatic dicarboxylic acid, such as α,ω-dicarboxylic acids (i.e., non-branched), branched dicarboxylic acid, cyclic dicarboxylic acids (e.g., cyclohexanedicarboxylic acid).

In some illustrative embodiments, the biodegradable polymer system comprises a first PEA polymer in which A is phenylalanine (Phe-PEA) and a second PEA polymer in which A is leucine (Leu-PEA). The ratio of Phe-PEA to Leu-PEA can be about 10:1 to about 1:1, or about 5:1 to about 2.5:1. Illustrative embodiments of degradable polymer blends in accordance with these aspects are described in U.S. Pat. No. 6,703,040 (Katsarava et al.).

Bioactive agent delivery systems comprising bioactive agent and polymeric material can be formulated by mixing one or more bioactive agents with the polymer(s). The bioactive agent can be present as a liquid, a finely divided solid, or any other appropriate physical form. Typically, but optionally, the bioactive agent delivery system will include one or more additives, such as diluents, carriers, excipients, stabilizers, or the like.

The particular bioactive agent, or combination of bioactive agents, can be selected depending upon one or more of the following factors: the application of the device (for implantation site), the medical condition to be treated, the anticipated duration of treatment, characteristics of the implantation site, the number and type of bioactive agents to be utilized, and the like.

The concentration of bioactive agent in a bioactive agent delivery system comprising bioactive agent and polymeric material can be provided in the range of about 0.01% to about 90% by weight, based on the weight of the final bioactive agent delivery system. In some aspects, the bioactive agent is present in the bioactive agent delivery system in an amount in the range of about 75% by weight or less, or about 50% by weight or less. The amount of bioactive agent in the bioactive agent delivery system can be in the range of about 1 μg to about 10 mg, or about 100 μg to about 5 mg, or about 300 μg to about 1 mg. The particular amount of bioactive agent can be selected based upon such factors as the condition to be treated, the particular bioactive agent(s) selected, patient parameters, anticipated duration of the implant within the patient, and the like factors. It is understood the amounts identified herein are illustrative only.

In some aspects of the invention, the bioactive agent delivery system can be utilized as a coating on a surface of the implantable device. In these aspects, all or a portion of the implantable device can be provided with the bioactive agent delivery system. Typically, at least a portion of the distal portion of the implantable device includes bioactive agent delivery system. In some aspects, the bioactive agent delivery system is provided in connection with the entire distal portion of the implantable device. In some aspects, the body member of the device includes a bioactive agent delivery system. It is understood that any desirable area of the distal portion can be provided with the bioactive agent delivery system.

In some embodiments, the bioactive agent delivery system is provided as a coating on a surface of the implantable device. The coating comprises one or more selected bioactive agents for delivery to the treatment site. Optionally, the coating can comprise polymeric material in addition to the bioactive agent(s).

Thus, in some aspects, the bioactive agent delivery system comprises a polymeric coating including bioactive agent and polymeric material. The polymeric coating can be coated from any suitable coating composition, and preferably is coated from a composition comprising a polymer and one or more suitable vehicles. Examples of such coating compositions include solutions, mixtures, emulsions, dispersions, blends, and the like that can be used to effectively coat a surface. In one embodiment, the coating composition comprises one or more bioactive agents and/or one or more polymers, and optionally one or more solvent vehicles.

The polymer coating can be formed from one or more coating compositions, or in one or more layers. The bioactive agents are either commixed with the polymeric coating, located between the polymeric coating and the device body member, or both commixed with the polymeric coating and located between the polymeric coating and the device body member. Additional bioactive agent(s) can optionally be located on the outside of the polymeric coating as desired for immediate delivery to the treatment site.

Bioactive agent system coatings can be applied to the implantable devices according to any suitable methods. For example, the coating can be applied by dipping, spraying, and other common methods for applying coatings to implantable devices. In some aspects, a surface of the implantable device can be pretreated prior to provision of a bioactive agent delivery system coating. Any suitable surface pretreatment commonly employed in coating implantable devices can be utilized in accordance with the invention, including, for example, treatment with silane, polyurethane, parylene, and the like.

In other aspects, the bioactive agent delivery system can be provided within a lumen of the implantable device. For example, when the body member of the device includes one or more lumens, any number of the lumens can be provided with a bioactive agent delivery system. When multiple lumens are included within the device, the bioactive agent delivery system can be the same or different within each individual lumen, as desired.

In still further aspects, a bioactive agent delivery system can be provided both as a coating on a surface of the implantable device and within a lumen of the implantable device. In accordance with these aspects of the invention, the bioactive agent delivery system can be the same or different for the coating and within the lumen.

The implantable device and bioactive agent delivery system are configured to provide release of a bioactive agent to a treatment site. In some aspects, the release of bioactive agent can be characterized as controlled release. As described herein, controlled release at the treatment site can mean control both in dosage (including dosage rate and total dosage) and duration of treatment. The mechanisms for controlled release of bioactive agent into the treatment site (e.g., vitreous) can include diffusion, osmosis, erosion, dissolution, and combinations of any of these mechanisms. The period for controlled release can be on the order of days to months.

In an embodiment of the present invention, the device delivers a therapeutically effective amount of bioactive agent, meaning that the device delivers an amount of a bioactive agent alone, or together with other substances (as described herein), that produces the desired effect (such as treatment of a medical condition such as a disease or the like, or alleviation of pain) in a patient. During treatment, such amounts will depend upon such factors as the particular condition being treated, the severity of the condition, the individual patient parameters including age, physical condition, size and weight, the duration of the treatment, the nature of the particular bioactive agent thereof employed and the concurrent therapy (if any), and like factors within the knowledge and expertise of the health practitioner. A physician or veterinarian of ordinary skill can readily determine and prescribe the effective amount of the bioactive agent required to treat and/or prevent the progress of the condition.

The term “treatment course” refers to the dosage rate over time of one or more bioactive agents, to provide a therapeutically effective amount to a patient. Thus, factors of a treatment course include dosage rate and time course of treatment (total time during which the bioactive agent(s) is administered).

The present invention is in one aspect directed to methods of using the device as described herein for effectively treating a treatment site within a patient's body, and in particular for delivering bioactive agents to a limited access region of a patient's body, such as the eye, ear, spinal cord, brain, joints, sinuses, glands, and the like.

In some aspects, the invention provides methods for delivering bioactive agent to a patient, the methods comprising steps of: (a) inserting a bioactive delivery device through tissue or membrane and into an implantation site comprising viscoelastic fluid or non-osseous tissue, and (b) maintaining the device at the implantation site to provide a therapeutically effective amount of the bioactive agent to the patient. The device comprises: (i) a nonlinear body member having a direction of extension, a longitudinal axis along the direction of extension, and a proximal portion and a distal portion, wherein at least a portion of the body member deviates from the direction of extension, (ii) a retention element at the proximal portion of the body member, the retention element configured to retain the implantable device at the implantation site, the retention element presenting an external profile of no greater than 0.5 mm when the device is implanted in a patient; and (iii) a bioactive agent delivery system at the distal portion of the body member, the bioactive agent delivery system comprising one or more bioactive agents.

Such methods in accordance with the present invention can advantageously be used to provide flexibility in treatment duration and type of bioactive agent delivered to the treatment site. In particular, the present invention has been developed for controllably providing one or more bioactive agents to a treatment site within the body for a desired treatment course. In one aspect of the present invention, the device body member and polymeric coating composition are configured to provide controlled release of a bioactive agent to a treatment site. As described herein, controlled release at the treatment site can mean control both in dosage (including dosage rate and total dosage) and duration of treatment.

Preferably, the configuration of the controlled release device provides one or more mechanical advantages, such as a built-in anchoring mechanism that reduces or prevents unwanted movement of the device within the body, reduced risk of unwanted ejection of the device from the body, and the like. Moreover, preferred embodiments of the invention can provide minimally invasive devices and methods for delivering one or more bioactive agents to a treatment site within the body. Accordingly, the invention can, in some embodiments, reduce risks of infection and complications associated with more invasive surgical procedures, as well as improve recovery time for patients requiring such treatments.

In preferred embodiments, the inventive device is easily retrievable from the body, such that the device is placed within the body only for the required treatment duration, and is removed upon completion of a treatment course. Preferably, the device provides enhanced durability of the coated composition, and thus the coated composition (minus the released bioactive agent) is removed from the implantation site upon completion of a treatment course. This can avoid potential harmful effects that could arise if one or more components of the device were left within the body beyond the treatment course (for example, if some of the coating is sheared off the device or otherwise delaminates from the device body member). In some aspects, configuration of the retention element can enhance retrievability of the device form the treatment site.

Optionally, the surface of the device body member can include surface configurations (for example, micro-etched surfaces, roughened surfaces, and the like), thereby improving the adhesion of a polymeric coating composition (when included) to the device body member surface and/or increasing the friction of the device body member relative to the tissue to improve anchoring of the device in the tissue.

In some aspects, the invention provides methods for treating a patient, the methods including administering a nonlinear implantable device as described herein, and permitting the implantable device to deliver bioactive agent to a treatment site within the patient's body. In an exemplary embodiment, the invention provides methods for treating ocular diseases or disorders.

Generally speaking, an incision is made in the body to provide access to an implantation site. For example, when used to delivery bioactive agent to the eye, an incision can be made in scleral tissue (sclerotomy). Conventional techniques can be used for the creation of a sclerotomy. Such techniques can include the dissection of the conjunctiva and the creation of pars plana scleral incisions through the sclera. Thus, in some aspects, the device can be driven into the vitreous through a sclerotomy previously made in the eye. Alternatively, the implantable device can be driven through the scleral tissue through a penetration in the scleral tissue (trans-sclera insertion) caused by a sharp distal end of the device.

The implantable device is inserted into the eye by rotating or twisting the device into the eye until the retention element rests within the desired tissue (e.g., the scleral tissue at the incision site). Once in place, the inventive devices present a low external profile that provides significant benefits over devices currently available for implantation in the eye.

The device can be inserted through tissue or membrane and into an implantation site comprising viscoelastic fluid or non-osseous tissue by rotating the device into the implantation site until the retention element rests within the tissue or membrane. When implanted in a patient eye, the device can be inserted through scleral tissue of a patient eye and rotated until the retention element rests within the scleral tissue. Alternatively, the device can be rotated until the retention element passes into a vitreous of the eye. Optionally, the retention element can be secured to scleral tissue via a suturing element. Such securement can be to scleral tissue of the eye. When the retention element is placed within the vitreous of the eye, the retention element can be secured to scleral tissue at a surface within the vitreous of the eye.

The device can remain at the implantation site a desirable amount of time to accomplish a treatment course. Optionally, the device can be removed from the implantation site after the treatment course is completed.

All percentages and ratios used herein are weight percentages and ratios unless otherwise indicated. All publications, patents and patent documents cited are fully incorporated by reference herein, as though individually incorporated by reference. Numerous characteristics and advantages of the invention meant to be described by this document have been set forth in the foregoing description. It is to be understood, however, that while particular forms or embodiments of the invention have been illustrated, various modifications can be made without departing from the spirit and scope of the invention.

What is claimed is:

1. An implantable device configured for implantation through tissue or membrane and into an implantation site comprising viscoelastic fluid or non-osseous tissue, the device comprising
(a) a nonlinear body member having a direction of extension, a longitudinal axis along the direction of extension, and a proximal portion and a distal portion, wherein at least a portion of the body member deviates from the direction of extension,
(b) a retention element at the proximal portion of the body member, the retention element configured to retain the implantable device at the implantation site, the retention element presenting an external profile of no greater than 0.5 mm when the device is implanted in a patient; and
(c) a bioactive agent delivery system at the distal portion of the body member, the bioactive agent delivery system comprising one or more bioactive agents.

2. The implantable device according to claim 1, the device configured for implantation into an eye.

3. The implantable device according to claim 1, wherein the nonlinear body member is a coil-shaped body member.

4. The implantable device according to claim 1, wherein the retention element comprises a lateral projection that projects at an angle from the direction of extension, the angle of projection being in the range of 40° to 100°.

5. The implantable device according to claim 4, wherein the lateral projection has a largest dimension substantially perpendicular to the direction of extension that is no more than 2 mm.

6. The implantable device according to claim 4, wherein the lateral projection has a height, measured along the longitudinal axis of the body member, of no more than 0.5 mm.

7. The implantable device according to claim 4, wherein the lateral projection is spaced from the body member a distance that corresponds to the thickness of the tissue or membrane through which the device is implanted.

8. The implantable device according to claim 4 wherein the lateral projection includes one or more suture holes.

9. The implantable device according to claim 1, wherein the retention element has a length, measured along the longitudinal axis, that is equal to or less than the thickness of the tissue or membrane through which the device is implanted.

10. The implantable device according to claim 1, wherein the retention element comprises a loop suitable for receiving a suturing element.

11. The implantable device according to claim 10, wherein the nonlinear body member has an outer diameter, and the loop of the retention element has a diameter that is less than half the length of the outer diameter of the nonlinear body member.

12. The implantable device according to claim 2, wherein the retention element is configured to be embedded within scleral tissue, when the device is implanted in a patient eye.

13. The implantable device according to claim 2, wherein the retention element is configured to be located within a vitreous of an eye and sutured against an inner scleral wall of the eye, when the device is implanted in a patient eye.

14. The implantable device according to claim 1 wherein the retention element comprises one or more openings provided along the proximal portion of the body member.

15. The implantable device according to claim 1 wherein the bioactive agent delivery system further comprises a polymeric matrix.

16. The implantable device according to claim 15 wherein the polymeric matrix comprises a first polymer and second polymer, the first polymer selected from polyalkyl(meth)acrylate, aromatic poly(meth)acrylate, or a combination of polyalkyl(meth)acrylate and aromatic poly(meth)acrylate, the second polymer comprising poly(ethylene-co-vinyl acetate).

17. The implantable device according to claim 1 wherein the body member includes a lumen, and wherein the bioactive agent delivery system is located within the lumen.

18. The implantable device according to claim 1 wherein the bioactive agent delivery system comprises a coating on at least a portion of the distal portion of the body member.

19. The implantable device according to claim 17 further comprising a bioactive agent delivery system as a coating on at least a portion of the distal portion of the body member.

20. A method for delivering bioactive agent to a patient, the method comprising steps of:
(c) inserting a bioactive delivery device through tissue or membrane and into an implantation site comprising viscoelastic fluid or non-osseous tissue, the device comprising:
(i) a nonlinear body member having a direction of extension, a longitudinal axis along the direction of extension, and a proximal portion and a distal portion, wherein at least a portion of the body member deviates from the direction of extension,
(ii) a retention element at the proximal portion of the body member, the retention element configured to retain the implantable device at the implantation site, the retention element presenting an external profile of no greater than 0.5 mm when the device is implanted in a patient; and
(iii) a bioactive agent delivery system at the distal portion of the body member, the bioactive agent delivery system comprising one or more bioactive agents,
(d) maintaining the device at the implantation site to provide a therapeutically effective amount of the bioactive agent to the patient.

21. The method according to claim 20 wherein the step of inserting a bioactive delivery device comprises inserting the device into a patient eye.

22. The method according to claim 20 further comprising a step of making an incision in the patient body prior to inserting the bioactive agent delivery device.

23. The method according to claim 20 wherein the step of inserting a bioactive delivery device through tissue or membrane and into an implantation site comprising viscoelastic fluid or non-osseous tissue comprises rotating the device into the implantation site until the retention element rests within the tissue or membrane.

24. The method according to claim 23 wherein the step of inserting a bioactive agent delivery device comprises inserting the device through scleral tissue of a patient eye and rotating the device until the retention element rests within the scleral tissue.

25. The method according to claim 23 wherein the step of inserting a bioactive agent delivery device comprises inserting the device through scleral tissue of a patient eye and rotating the device until the retention element passes into a vitreous of the eye, and securing the retention element to scleral tissue at a surface within the vitreous of the eye.

26. The method according to claim 20 further comprising passing a suture element through a portion of the retention element, and suturing the device to the tissue or membrane.

27. The method according to claim 20 further comprising a step of removing the device from the implantation site after a desired treatment course.
